(19) United States
(12) Patent Application Publication
(10) Pub. No.: US 2016/0045755 A1
(43) Pub. Date: Feb. 18, 2016

(54) APPARATUS, SYSTEM AND METHOD FOR TREATING FAT TISSUE

(71) Applicants: Donghoon CHUN, Sunnyvale, CA (US); Daniel E. FRANCIS, Mountain View, CA (US); Jessi E. JOHNSON, Sunnyvale, CA (US); Steven W. KIM, Los Altos, CA (US); Kevin LE, East Palo Alto, CA (US); Ted Y. SU, Sunnyvale, CA (US)

(72) Inventors: Donghoon CHUN, Sunnyvale, CA (US); Daniel E. FRANCIS, Mountain View, CA (US); Jessi E. JOHNSON, Sunnyvale, CA (US); Steven W. KIM, Los Altos, CA (US); Kevin LE, East Palo Alto, CA (US); Ted Y. SU, Sunnyvale, CA (US)

(21) Appl. No.: 14/829,434

(22) Filed: Aug. 18, 2015

Related U.S. Application Data
(60) Provisional application No. 62/038,627, filed on Aug. 18, 2014.
(60) Provisional application No. 62/134,452, filed on Mar. 17, 2015.
(60) Provisional application No. 62/191,106, filed on Jul. 10, 2015.

(51) Int. Cl. A61N 1/40; A61F 7/00

(52) U.S. Cl. CPC A61N 1/40; A61F 7/00; A61F 2007/0056; A61F 2007/0095

(57) ABSTRACT

Systems and methods are provided for use in the reduction of fat, which may include a number of features. In some embodiments, the systems and methods may include an applicator having electrodes that form capacitors to heat tissue, an acquisition chamber having a cooling system, and a vacuum input providing a vacuum to acquire tissue to be treated.

### CROSS REFERENCE TO RELATED APPLICATIONS

This application claims the benefit of U.S. Provisional Patent Application No. 62/038,627 filed Aug. 18, 2014 titled “Apparatus and Methods for Treating Fat Tissue”; U.S. Provisional Patent Application No. 62/134,452 filed Mar. 17, 2015 titled “Apparatus and Method for Treating Fat Tissue”, and U.S. Provisional Patent Application No. 62/191,106 filed Jul. 10, 2015, titled “Apparatus and Methods for Treating Fat Tissue,” the disclosures of which are incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure relates generally to non-invasive apparatus, systems and methods to heat tissue to reduce fat volume in a subject.

### BACKGROUND

Forms of fat reduction by applying energy to fat cells to cause necrosis have been proposed. In particular heating tissue with electromagnetic energy and using diathermy have been proposed to reduce fat cells.

### SUMMARY OF THE DISCLOSURE

An applicator for use in the reduction of fat is provided, the applicator comprising a housing containing a tissue acquisition chamber having an internal surface adapted to contact tissue, a cooling plate, wherein the cooling plate forms at least a portion of the internal surface of the acquisition chamber, a coolant chamber adjacent at least a portion of the cooling plate, at least one electrode pair comprising electrodes positioned on opposing sides with respect to the tissue acquisition chamber, and a tissue acquisition structure configured to bring tissue into the tissue acquisition chamber.

The electrodes may be separated from the acquisition chamber by the cooling plate. The electrodes may form at least a portion of the internal surface of the acquisition chamber. The cooling plate comprises a cutout and wherein an electrode of the electrode pairs is positioned within the cutout. The electrodes may be separated from the cooling plate by the coolant chamber.

The coolant chamber may include a cooling fluid moving through the coolant chamber. The electrodes may be coupled to a wall of the cooling plate opposite the wall which forms the internal surface. The coolant chamber may be positioned adjacent the cooling plate and the electrodes.

The tissue acquisition chamber may comprise a tissue acquisitions structure configured to bring tissue into the acquisition chamber. The tissue acquisition structure may include vacuum tube configured to be coupled to a vacuum source and connected to the tissue acquisition chamber.

Electrodes may comprise a coolant channel formed therein and configured to receive a coolant flowing through the coolant channel. Electrodes forming a first of the at least one electrode pairs may be substantially parallel with respect to each other. The electrodes of the first electrode pair may form an angle with respect to each other of between 45 and 0 degrees.

At least one of the electrodes may be movable within the housing from a first configuration to a second configuration. The second configuration may comprise an angular orientation with respect to the first configuration. The electrodes may comprise a plurality of electrode pairs. The electrodes may further comprise a second pair of electrodes, wherein the electrodes of the first electrode pair have a first dimension and the electrodes of the second electrode pair have a second dimension.

The dimension may comprise one or more of length, width size, shape, surface area, and volume. The acquisition chamber internal surface may comprise a first side and an opposing second side, and a third side and an opposing fourth side, wherein the plurality of electrode pairs comprises a first electrode pair including a first electrode positioned adjacent the first side of the acquisition chamber and a second electrode positioned adjacent the second side of the acquisition chamber, and wherein the second electrode pair comprises a third electrode positioned adjacent the third side of the acquisition chamber and a fourth electrode positioned adjacent the fourth side of the acquisition chamber.

The tissue acquisition chamber may form a tissue treatment zone when tissue is acquired into the chamber. The electrodes may be positionable to form a therapeutic treatment zone of tissue acquired within the acquisition chamber. The applicator may comprise a plurality of coolant chambers. Each of the plurality of coolant chambers may be separate from the other of the coolant chambers.

The applicator may further comprise a sensor configured to sense a temperature of tissue outside of the treatment zone. The applicator may comprise one or more sensors configured to sense contact of skin with the acquisition chamber internal surface at one or more locations on the acquisition chamber internal surface.

At least a portion of the acquisition chamber inner surface may comprise a flexible material configured to conform to tissue when it is acquired into the chamber. The internal surface of the acquisition chamber may comprise a first side and second opposing side opposing the first side wherein the first and second sides form an angle with respect to each other of greater than 0 degrees. The internal surface of the acquisition chamber may comprise a first side and second opposing side opposing the first side wherein the first and second sides form an angle with respect to each other of greater than 45 degrees. The acquisition chamber internal surface may comprise a first side and an opposing second side, and a third side and an opposing fourth side, wherein the plurality of electrode pairs comprise a first electrode positioned adjacent the first side of the acquisition chamber and a second electrode positioned adjacent the second side of the acquisition chamber, wherein the first side forms a first angle with respect to the second side and the third side forms a second angle with respect to the fourth side, wherein the second angle is greater than the first angle. The first angle may be less than or equal to 45 degrees.

According to variations, a system for use in the reduction of fat is provided comprising a housing containing a tissue acquisition chamber having an internal surface adapted to contact tissue; a cooling plate, wherein the cooling plate forms at least a portion of the internal surface of the acquisition chamber, a coolant chamber adjacent at least a portion of the cooling plate, at least one electrode pair comprising electrodes positioned on opposing sides with respect to the tissue acquisition chamber, and a tissue acquisition structure configured to bring tissue into the tissue acquisition chamber; and a control console comprising a coolant source configured to circulate a coolant through the coolant chamber, an RF generator configured to be coupled to the plurality of electrodes, and a controller configured to control the delivery of an RF signal to the plurality of electrodes.

The at least one electrode and tissue acquisition chamber may form a tissue treatment zone when tissue is acquired into the chamber where the applicator further comprises a sensor configured to sense a temperature of tissue outside of the treatment zone and wherein the controller is configured to determine when the temperature is above a threshold temperature. At least one coolant chamber may further form the tissue treatment zone.

At least one contact sensor may be configured to sense contact of tissue with the internal surface of the acquisition chamber at least one location on the acquisition chamber wherein the controller is further configured to determine when adequate tissue contact with the cooling plate is in place.

A generator of the system may supplies an RF signal between 1 MHz and 500 MHz; between 5 and 350 MHZ or between 5 MHz and 50 MHz to the electrodes.

According to variations, a method of body contouring is provided comprising moving tissue comprising skin and fat into an acquisition chamber of an applicator, heating a therapeutic zone of tissue positioned with in the chamber at a temperature within a target temperature range for a period of time using electrodes positioned on opposing sides of the acquisition chamber in the applicator, altering tissue temperature outside of the therapeutic zone to a temperature below the target temperature range.

An RF signal may be supplied to the electrodes from and RF generator at a frequency of between 1 MHz and 500 MHz.

The method may further comprise the step of determining when tissue has made adequate contact with an internal surface of the acquisition chamber.

The method may further comprise determining when the temperature of tissue at a location outside of the therapeutic zone is higher than a threshold temperature.

The method may further comprise the step of reducing energy supplied to the electrodes from the RF generator in response to determining the temperature of tissue at a location outside of the therapeutic zone is higher than a threshold temperature.

The method may further comprise: changing at least one configuration of one or more of the electrodes to treat a first volume of tissue within the therapeutic zone a different amount than a second volume of tissue within the therapeutic zone.

The step of changing at least one configuration may comprise changing the angle of orientation between two electrodes forming an electrode pair. The electrodes may form more than one electrode pairs.

The step of using electrodes may comprise using more than one electrode pair to treat tissue within the therapeutic zone.

The method may further comprise supplying a first electrical signal having a first parameter to a first electrode pair to treat a first volume of tissue within the therapeutic zone, and supplying a second electrical signal having a second parameter to a second electrode pair to treat a second volume of tissue within the therapeutic zone a different amount than the first volume of tissue.

According to variations, a method of body contouring is provided that comprises bringing tissue comprising skin and fat into an acquisition chamber of an applicator, contouring a volumetric size and shape of a therapeutic zone of tissue by selectively heating and cooling using at least one electrode pair positioned in the applicator on opposing sides of the acquisition chamber.

The step of selectively heating may comprise varying one or more electrical parameter of energy delivered to the tissue through the at least one electrode pair. The step of varying one or more electrical parameter may comprise altering one or more electrode pair orientation. The step of varying one or more electrical parameter may comprise selectively employing a plurality of electrode pairs. The step of selectively cooling may comprise altering one or more of flow rate and temperature of a coolant altering temperature of tissue at one or more location in the acquisition chamber.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an isometric external view of an applicator according to the present invention placed on human layers of skin and fat. The applicator in this iteration has an arch-shaped form factor.

FIG. 2 is an isometric view of the applicator of FIG. 1 according to the present invention placed on human layers of skin and fat, also showing two planes used for the following cross-sectional views are shown.

FIG. 3 is a cross sectional view of the applicator according to the present invention cut along the Front Plane of FIG. 2. In this image, the applicator has fully acquired skin and fat volume, using the suction from the vacuum tube.

FIG. 4 is a cross sectional view of the applicator according to the present invention cut along the Right Plane of FIG. 2. In this image, the applicator has fully acquired the skin and fat layer using suction from the vacuum tube.

FIG. 5 is an isometric view of an applicator according to the present invention with the housing layer removed. This iteration of the applicator makes use of two shapes of electrodes and a total of four electrodes overall.

FIG. 6 is an isometric view of an applicator according to the present invention with the housing layer removed. This iteration of the applicator shows two different sized rectangular electrodes and has a total of four electrodes overall.

FIG. 7 is an isometric external view of an applicator according to the present invention place on human layers of skin and fat. The applicator in this iteration has an elliptical dome and form factor.

FIG. 8 is an isometric view of the applicator of FIG. 7 according to the present invention with the housing layer removed. This iteration has two identical electrodes in a rectangular shape formed onto the dome.

FIG. 9 is an external view of the applicator of FIG. 7 according to the present invention. Also shown are the two planes used to cut the applicator for the following figures and cross-sectional views.

FIG. 10 is a cross-sectional view of the applicator according to the present invention cut with the Right plane of FIG. 9. This view is taken after the applicator has fully acquired the skin and fat layers.

FIG. 11 is an external view of an applicator according to the present invention on human layers of skin and fat. This iteration has a trapezoidal form factor. Two planes that were used for the following cross-sectional views are also shown.

FIG. 12 is a cross-sectional view of the applicator of FIG. 11 according to the present invention cut with the Right Plane.

FIG. 12A shows an angle of orientation between electrodes of the electrode pair of FIG. 12.

FIG. 13 is a cross-sectional view of the applicator of FIG. 11 according to the present invention cut along the Front Plane.

FIG. 14A is a cross-sectional view of an applicator according to the present invention wherein a first orientation of the electrodes with respect to the cooling plates is illustrated.

FIG. 14B is a cross-sectional view of an applicator according to the present invention wherein a second orientation of the electrodes with respect to the cooling plates is illustrated.

FIG. 15A is a cross-sectional view of an applicator according to the present invention wherein the electrode sits directly on the cooling plate.

FIG. 15B is a cross-sectional view of an applicator according to the present invention wherein sits directly adjacent skin in the acquisition chamber.

FIG. 16A is a cross-sectional view of an applicator according to the present invention wherein multiple electrodes are positioned on each side of the acquisition chamber in a first configuration.

FIG. 16B is a cross-sectional view of the applicator of FIG. 16A wherein multiple electrodes are positioned on each side of the acquisition chamber in a second configuration.

FIG. 16C is a cross-sectional view of an applicator according of FIG. 16A wherein multiple electrodes are positioned on each side of the acquisition chamber in a third configuration.

FIG. 16D is a schematic cross-sectional view of electrodes of the applicator of FIG. 16A in a fourth configuration.

FIG. 16E is a schematic cross-sectional view of electrodes of the applicator of FIG. 16A in a fifth configuration.

FIG. 16F is a schematic cross-sectional view of electrodes of the applicator of FIG. 16A in a sixth configuration.

FIG. 17 is an isometric external view of an applicator according to the present invention showing two planes used for the following cross-sectional views.

FIG. 18 is an isometric cutaway view of a first side of the applicator of FIG. 17 according to the present invention.

FIG. 19 is an isometric cutaway view of an opposing side of the applicator of FIG. 18 according to the present invention.

FIG. 20 is a cutaway elevational view of an end of the applicator of FIG. 17.

FIG. 21 is a cross sectional view of the applicator of FIG. 17 cut along the Front Plane of FIG. 17.

FIG. 22 is a cross sectional view of the applicator of FIG. 17 cut along the Right Plane of FIG. 17.

FIG. 23 is a bottom isometric view of the applicator of FIG. 17.

FIG. 24 is a bottom isometric view of the applicator of FIG. 17.

FIG. 25 is a bottom elevational view of the applicator of FIG. 17.

FIG. 26 is a schematic illustration of a housing of an applicator.

FIG. 27 is a schematic illustration of a system for reducing fat volume in a subject including a control console and applicator.

### DETAILED DESCRIPTION

Apparatus systems and methods of body contouring are provided. Accordingly, apparatus, systems and methods are provided to non-invasively heat tissue to reduce fat volume in a subject. It is believed that prolonged exposure to elevated temperatures is likely to cause fat cell necrosis, which is expected to lead to a reduction in fat volume when the necrotic fat cells are then absorbed by the body. Subcutaneous fat cells in targeted areas of the body (“treatment zone” or “therapeutic zone”) may be heated to a predetermined temperature, a minimum temperature or within a predetermined range of temperatures, to thermally damage the cells.

According to variations, parallel plate capacitive diathermy may be used to heat tissue between capacitor plates or electrode pairs. In accordance with the fat reduction device of the invention, the patient's tissue, including skin (note that, as used herein, “skin” refers to the dermis and epidermis and does not include the hypodermis) and fat is placed between the electrodes and energy is delivered to the tissue through the skin. The plate or electrode angles may also be arranged to orient the direction of the fields passing through the skin and/or fat. The plates or electrodes may be oriented such that the electric fields pass through the skin surface at an angle which is substantially or sufficiently perpendicular to the surface of the skin. The capacitor plates may be fully parallel or may form angles up to 45 degrees with respect to each other. According to further variations, the tissue and/or electrodes are positioned such that desired energy in the electric field between the plates is absorbed in the fat.

According to some variations, a fat reduction apparatus and/or system are provided comprising a patient-contacting applicator. An applicator in accordance with iterations the invention may use a tissue acquisition structure to draw tissue into an acquisition chamber to position it for treatment. Another device such as a pinching device may be used to draw tissue into a chamber. Vacuum may be used to draw the tissue into a vacuum chamber. One or more electrode pairs may be positioned on opposing sides of the applicator vacuum chamber and may deliver energy through acquired skin to heat the tissue through dielectric heating or diathermy.

According to variations, the electrodes are movable or positionable to vary selected energy delivered to specific locations. According to variations, a plurality of electrode pairs may be provided. Each of the pairs of electrodes and/or parameters of an electrode pair may be individually selected for desired treatment and/or desired contouring effect. The electrode pairs may or may not have different sizes and/or shapes and may be movable to change orientation.

According to variations, a user may select one or more electrode orientations of one or more electrodes to vary the energy delivered to one or more locations so as to remove desired select amounts from selected regions and thereby contour the subject's body at a selected location.

According to variations, a plurality of electrode pairs may be provided on the applicator. Different energy delivery parameters may be provided at each electrode location. Such delivery parameters may include, for example, frequency, duration, power, waveform, and/or electrode orientation. According to variations, a method of contouring is provided comprising delivering selected treatment parameters to a plurality of selected locations. According to variations, the electrode pairs may be movable to provide varying electrode orientations and varying delivery of energy field strengths and/or location.

According to variations, the electrode pairs may have different sizes, lengths widths, surface areas (“dimensions” as used herein). Varying electrode dimensions and orientations (“configurations”) as used herein may deliver different heating to different areas of fat, thus allowing for specific contouring of the area by a user.

In embodiments of the invention, the vacuum chamber inner walls may be angled to facilitate the acquisition of tissue. According to variations, tissue may be positioned between the electrode pairs located on the applicator in a parallel or substantially parallel configuration with respect to each other. The electrodes attached to the applicator may have a maximum angle of orientation with respect to each other. For example such maximum angle may be about 45 degrees. In embodiments of the invention, the electrodes may be oriented to be substantially parallel to the inner surface walls of the acquisition or vacuum chamber and thus with the skin surface.

An active cooling system may be used to protect the upper region of the skin, including the dermis, from excessive heating. The cooling system may protect the entire surface of the skin, into the deep dermis, both in the region of treatment and in regions where treatment may result in hot spots. Such cooling system may, for example comprise a cooling plate coupled to or in contact with the skin to cool the skin. According to iterations of the invention at least a portion of the vacuum chamber inner wall is formed of the cooling plate. A coolant such as water may be circulated to and from a chiller through a cooling chamber adjacent the cooling plate. According to other iterations of the invention, the vacuum chamber inner walls are positioned adjacent a cooling plate and the electrode pairs may be separated from the cooling plate by a coolant flowing through the cooling chamber. According to iterations, the cooling chamber or chambers may be provided with features to distribute and route cooling fluids evenly over the cooling plate surface.

In one structure, an arrangement of layers may include a first capacitor plate, a cooling fluid, cooling plate, epidermis, dermis, fat region, dermis, epidermis, cooling plate, cooling fluid and a second electrode. The coolant may further be used to cool one or more electrodes. In embodiments of the invention, the face of the electrode may be positioned on the interior wall of the cooling plate with the cooling fluid flowing behind the electrode and cooling plate.

Flow channels in electrodes may be provided to ensure coolant is uniformly flowing across the electrode surface. In embodiments of the invention, the face of the electrode may be coated with a thin insulator to prevent it from contacting the cooling fluid, which may prevent corrosion and/or shorting.

In embodiments of the invention, the cooling plates may be formed of, for example a plastic or ceramic material. The cooling plate in one example is formed of a 0.040″ thick PETG sheet that is vacuum formed to create the shape of the vacuum chamber.

In embodiments, the cooling plate or dielectric forms a larger surface area than the surface area of the electrodes. Increasing the surface area cooled may allow the temperature at the target tissue to be increased. In embodiments of the invention the cooling may be extended to include all of the tissue captured in the applicator, by, for example, forming the interior of the applicator of a plastic or ceramic material and including a cooling fluid channel behind the entire interior wall of the applicator. According to one aspect a coolant may circulate over the cooling plate in a flow path defined by one or more inlets, one or more chambers and one or more outlets. Various features of the applicator, housing, cooling plate, and/or electrodes may define the chamber or chambers and/or flow path or paths. The coolant may further be used to cool one or more electrodes or electrode pairs. In embodiments of the invention the applicator may include multiple cooling channels or chambers wherein cooling fluid in the different chambers may be cooled to different temperatures. In embodiments of the invention, cooling chambers may be positioned to provide additional cooling in the region of known hot spots.

According to variations, decreasing the temperature of the cooling fluid may allow the temperature at the target tissue to be increased. In embodiments of the invention, the coolant or cooling fluid may have a temperature above freezing to a temperature below the target range for the therapeutic zone. Thus cooling may include a fluid that is wanner than room temperature or skin temperature but is nonetheless cooler than target or therapeutic temperatures or ranges. The cooling temperature of the fluid, for example, may be selected by the controller, may be based on patient or sensed temperature or hot spot identification, or may depend on the treatment protocol including the amount of energy delivered or specific locations for cooling for purposes of body contouring.

In embodiments of the invention the cooling may be in-line active cooling where the temperature of the cooling fluid is modulated according to specific feedback criteria, such as, for example, the differential temperature between the cooling fluid at the applicator inlet and the cooling fluid temperature at the cooling fluid outlet.

In accordance with iterations of an applicator herein, the applicators are shaped in a manner that provides adequate tissue acquisition into the chamber and surface contact with the cooling plate while providing desired energy delivery with a parallel or near parallel orientation of the electrodes of the electrode pairs with respect to each other.

One or more electrode pairs, electrode configuration, or electrical parameters may be utilized during a procedure. A signal generator switch may be used to select an electrode pair or electrode pairs and/parameters or configuration of a selected pair during a procedure. The use of the electrode pairs may be alternated during a procedure and may be selected according to a predetermined algorithm or in response to patient feedback or one or more conditions sensed by one or more sensors. According to iterations, the electrodes may be movable during treatment or between treatment cycle to provide for a desired heating of a volume of tissue.

An applicator may be used with a control console. The console components may comprise one or more signal generators and amplifier to deliver energy at a desired frequency. While a range of frequencies and power levels may be used for the present treatment optimal frequency and power settings will maximize the transfer of energy to the fat without overheating. In some embodiments, the frequency is between 1 MHz and 500 MHz. In some embodiments the frequency is between 1 MHz and 400 MHz. In some embodiments, the frequency is between 5 MHz and 350 MHz. In some embodiment the desired frequency is between 5 MHz and 50 MHz. The desired frequency may be 10 and 50 MHz or around 27 MHz. The frequency may be selected or modified prior to or during any treatment cycle. The power is selected to provide optimal heating and may depend on a variety of parameters including but not limited to, for example, the volume of tissue, the duration, frequency, or waveform of energy delivery, electrode features or configurations and/or cooling parameters. In embodiments of the invention, the wave shape of the energy may be modified to optimize patient safety and/or efficacy. In embodiments of the invention the wave shape may be a sine wave or square wave. In embodiments of the invention, the energy may be transmitted in pulses.

According to the device, systems and methods of the invention, fat reduction in a subject is provided in a manner that results in a desired contouring effect. While numerous possibilities exist, in some embodiments, for example, fat removal may be transitioned at the edges of a treatment zone. In other instances, for example, in some embodiments uniform fat reduction throughout the entire treatment zone is provided. In other instances, for example in some embodiments fat removal may be shaped in therapeutic zones.

According to variations, the electrodes may be movable or positionable to vary energy delivery concentrations to specific locations. According to variations, the angle of orientation of an electrode of an electrode pair may be varied to deliver an energy field having different strengths and/or to specific fat target areas. According to variations, a user may select one or more electrode orientations to vary the energy delivered to one or more locations so as to remove desired select amounts of fat from selected regions and thereby contour the subject's body at a selected location.

According to variations, a plurality of electrode pairs may be provided. Each of the pairs of electrodes may be individually selected for desired treatment and/or desired contouring effect. According to variations, the electrodes may have different sizes, lengths widths, surface areas (“dimensions” as used herein). According to variations, energy delivery parameters of one or more electrode pairs may be individually selected for desired treatment and/or desired contouring effect. Such energy delivery parameters may include, for example, frequency, duration, power, waveform, and/or electrode orientation.

According to variations, a method of contouring is provided comprising delivering selected treatment parameters to a plurality of selected locations. Varying electrode dimensions and orientations (“configurations”) as used herein may deliver different heating to different areas of fat, thus allowing for specific contouring of the area by a user.

According to variations, the temperature, flow path or dynamics, and/or location of the coolant may be used to direct heating to the target or therapeutic zone or reduce heating in not target or therapeutic zones and thus may be used to further contour the tissue.

According to variations, to optimize energy delivery into the fat tissue, the electrical circuitry may be impedance matched to the cooling and captured tissue. Impedance matching may be done prior to treatment, by, for example, using a tuning circuit connected to the electrodes and tuning it to match the tissue load. Alternatively an auto-tuning circuit may be used to automatically sense load mismatches and to tune to maximize energy transfer and minimize loss.

The console may also include a vacuum pump. The vacuum pump and/or applicator may include sensors to insure proper vacuum pressure in the vacuum chamber. The console may also include a chiller/heater and coolant pump.

Skin Protection:

Sensors or other feedback may be used in a procedure, for example to ensure proper skin acquisition and contact with the applicator as well as to ensure desired heating and temperatures. For example, temperature of the skin or the cooling plate may be sensed. Specific locations on the cooling plate or of the skin may be monitored. Additionally, potential hot spots induced by the electrode configuration and/or electrode pair interactions may be monitored for overheating. Hot spots are generally undesired heating outside of a desired therapeutic zone. In particular some hot spots may be considered, to be such locations where the temperature is greater than the peak desired temperature which resides in the therapeutic zone. Skin contact on dielectric or cooling plate may also be monitored with sensors to ensure full or desired contact with the treatment chamber and thus optimal or desired cooling of the skin or heating pattern of the tissue.

According to one variation, to protect the skin, sensors along the interior surface of the chamber are used to detect tissue acquisition and contact. Examples of sensors and methods to ensure proper tissue acquisition may include but are not limited to the following set forth below. With respect to tissue acquisition, according to some variations, all or most of the skin may be in contact with the cooling plate.

Thermocouples or fiber optic temperature sensors may be used to ensure adequate tissue acquisition. A thermocouple or fiber optic temperature sensor positioned inside of the tissue acquisition chamber may be used to detect tissue acquisition based on temperature increase or decrease when acquired tissue contacts the sensor at the thermocouple location, at which point the sensed temperature may be higher or lower than a baseline temperature for example, that is either higher or lower than skin temperature. According to iterations, multiple thermocouples or fiber optic temperature sensors may be placed in a plurality of locations or areas for tissue acquisition. A controller may determine full tissue acquisition based on tissue acquisition sensed at multiple locations.

IR reflective sensors may be used to ensure desired tissue acquisition. For tissue acquisition sensing, the sensor may reflect light off of the human skin. As the vacuum pulls the skin and fat into the tissue acquisition chamber, the tissue may get closer to the sensors which may send signals to a microcontroller. In the software of the microcontroller, a threshold may be set to signify when the tissue is in contact with the tissue acquisition chamber wall. With a plurality of the reflective sensors placed in positions believed to be the last locations to be filled during tissue acquisition (such as the top of the tissue acquisition chamber), the microcontroller can identify when all of the sensors have crossed the threshold and conclude that full tissue acquisition has been achieved.

IR temperature sensors may also be used to ensure desired tissue acquisition. The temperature sensors may also be non-contacting sensors and if desired, located outside of the vacuum or acquisition chamber. Similar principles may be used to measure the temperature of an object in its field of view as are with the IR sensors. According to variations the acquisition chamber may be configured so that tissue is not in the sensor's field of view and the sensor may then measure ambient temperature. Once the tissue is fully acquired, in such location, the sensor may then measure which should be higher than ambient temperature. According to a variation of the invention, a controller receives signals from a plurality of temperature s sensors positioned at various locations to identify when tissue is in contact with the chamber or is fully acquired.

Vacuum pressure sensors may also be used to ensure desired tissue acquisition and contact with acquisition chamber. In accordance with iterations, the tissue acquisition chamber may include a pressure sensor and a valve having a small leak until the tissue is fully acquired. For example, a push-button valve may be placed inside the tissue acquisition chamber in a location where tissue is believed to be acquired last. Once the tissue is fully acquired, it will push the button that closes the valve and allows the vacuum pressure sensor to detect the amount of pressure that the vacuum pump is producing. A controller reading a signal from the vacuum sensor may determine or indicate that full tissue acquisition has occurred inside of the tissue acquisition chamber when the know pressure of the vacuum pump is sensed. If the tissue is not fully acquired, it will not close the valve and the sensor will detect a pressure different than the known pressure of the vacuum pump in which case the controller will determine or indicate tissue is not fully acquired. Alternatively, a push-to-open valve may be used. According to this iteration, the vacuum pressure sensor coupled to the tissue acquisition chamber detects only atmospheric pressure as long as the valve remains closed, until the tissue is fully acquired, whereupon it pushes the button that opens the valve and the sensor sense a pressure equal to the pressure produced by the vacuum pump. The pump may also include a pressure sensor where the sensed pressure at the pump may be compared to the sensed pressure in the acquisition chamber to determine if there is a leak.

Piezoelectric contact sensors may also be used to ensure desired tissue acquisition by measuring physical contact with the skin.

The shapes of the electrodes and/or associated dielectric material between the electrodes and the skin may be further modified to protect the skin surface from fields which may cause hot spots and/or burns. In one embodiment, adjacent electrodes may be spaced from each other sufficiently to prevent or avoid hot spots that may develop in part due to electrical interaction of the adjacent electrode. In other embodiments, the electrodes have rounded edges to reduce high field strengths or current densities. In embodiments of the invention, shields may be placed at the edge of an electrode to protect skin outside the electrode edges. In embodiments of the invention, absorbers may be placed at the edge of the electrodes to protect skin outside the electrode edges. In embodiments of the invention, the electrode could be shaped to wrap around the tissue in order to confine the electric fields to the target tissue. In embodiments of the invention, a series of holes (e.g., “Swiss cheese” holes) may be used to reduce the fields radiated at certain locations, thus protecting the tissue at such locations.

A key feature of the present invention is the protection of the epidermis and dermis, particularly in regions outside the tissue held between the electrode plates. Areas of the dermis and epidermis which are not parallel to the capacitor plate surface are particularly susceptible to electric fields which couple excess energy into the dermis and epidermis, resulting in hot spots which may lead to tissue damage. This may be particularly true where the electric fields emitted by the electrodes are parallel to the skin surface. In an applicator where the tissue is pinched into place or pulled into a chamber to be properly positioned, regions of the skin will naturally be at angles which would result in parallel or substantially parallel electric fields and, thus, potential hot spots. According to variations of the invention, such an electrode may be spaced or define a gap between an adjacent edge of the electrode and a location on the applicator where the skin surface is oriented generally or approximated perpendicular to the electrode or parallel to the electric field created by an energized electrode.

Target Tissue Temperature Control

A key feature of the invention is the ability to uniformly heat fat tissue to an optimal temperature and hold the tissue at that temperature for an extended period of time, such as, for example between 10 and 90 minutes, between 15 minutes and 60 minutes. According to iterations of the invention, the fat cells are heated to a temperature or within a temperature range for both patient comfort and treatment effect. The tissue is heated to a temperature of between 39 and 60 degrees or higher. In some embodiments, the tissue may be heated to a temperature of between 44 and 50 degrees. In some embodiments, the tissue may be heated to a temperature of between 43 and 45 degrees. According to some embodiments of the invention at least 40%, at least 50% or at least 60% of the target fat tissue is heated to a temperature within the desired temperature range.

According to one variation, power delivered may be modulated according to the patient's feedback, by, for example, increasing the power delivered provides feedback of sensation of discomfort and then reducing the power until the discomfort goes away. In embodiments of the invention, the energy may be delivered in pulses and the power delivered modulated by modulating the frequency and/or power of the pulses. Once an appropriate power level is reached, it can be maintained for the duration of the treatment. Temperature sensors may be used as an alternative or in addition to patient feedback. An alternative algorithm may predict temperature of the target tissue by connecting the skin surface tissue to the temperature at the target tissue, using, for example, the amount of tissue pinched or captured in the applicator chamber as a variable. Other variables might include power, time, or coolant temperature.

A variety of temperature sensing feedback and control mechanisms may be used, for example, skin temperature may be measured as an indicator of target tissue temperature. Fiber optic temperature sensors may be used to measure skin temperatures. A thin needle may be implanted and used, for example, with a thermocouple or fiber optic infrared sensor to measure the temperature of the target tissue. Microwave radiometry may be used to measure the temperature of the target tissue, including, using a phased array antenna to detect the temperature at depth. One or more of the sensors used in the applicator described herein may use microwave radiometry. An infrared sensor may be used to measure the temperature of the skin surface. Changes in tissue impedance as the target tissue is warmed, may be measured by passing a signal having an alternate frequency through the capacitor plates and measuring change in impedance. In one embodiment, this may involve the use of multiple electrodes on or near the skin.

In one embodiment, the frequency may be swept and changes in the resonant frequency and/or Q of the captured skin and fat measured. Changes in AC resistance/conductivity of the captured tissue may be used to determine temperature. Materials may be implanted in the target tissue region which provide indications of tissue temperature, such as, for example: materials which change in color, where the color change is visible through the skin surface; materials which change in phase, where such changes may be measured by, for example using ultrasound, such as, for example, an ultrasonic flow meter, to detect the phase change; and microbubbles injected into the skin. Ultrasound transducers may be used to measure changes in transmission time as the target tissue heats up.

Tissue temperature in the target region may also be measured by looking at bio-markers (e.g. histamines, proteins and/or white blood cell count) in the patient's blood, bodily fluids and/or breath. The bio-markers would be indicative of the amount of damage done to the target tissue, allowing the operator or system to detect the damage and stop or reduce the energy if it was greater than desired. The patient's breath may be analyzed using, for example, a gas chronograph, such as, for example, the “Z—Nose” electronic sensor. Other changes in the characteristics of the target tissue might also be used as feedback mechanisms.

In variations of the invention, the electrodes forming the capacitor plates may be positioned to optimize even heating of the fat which is captured in the applicator chamber. In variations of the invention the electrodes forming the capacitor plates may be at a fixed angle to the surface of the cooling plate which forms the inner surface of the acquisition chamber. In variations of the invention the electrodes forming the capacitor plates may be adjusted prior to initiating treatment and held fixed during treatment. Alternatively, the electrodes may be adjusted during the treatment. In variations of the invention the electrodes forming the capacitor plates may be adjustable such that they may be positioned at various angles to the surface of the cooling plate which forms the inner surface of the acquisition chamber. In variations of the invention, heating patterns may be evened out using multiple sets of smaller electrodes which may be angled independently and spaced for optimal heating. In variations of the invention adjustable electrodes and multiple electrodes may be used to customize field strengths in the tissue in order to achieve, for example, custom contouring of the fat tissue without creating hot spots or burns in the skin.

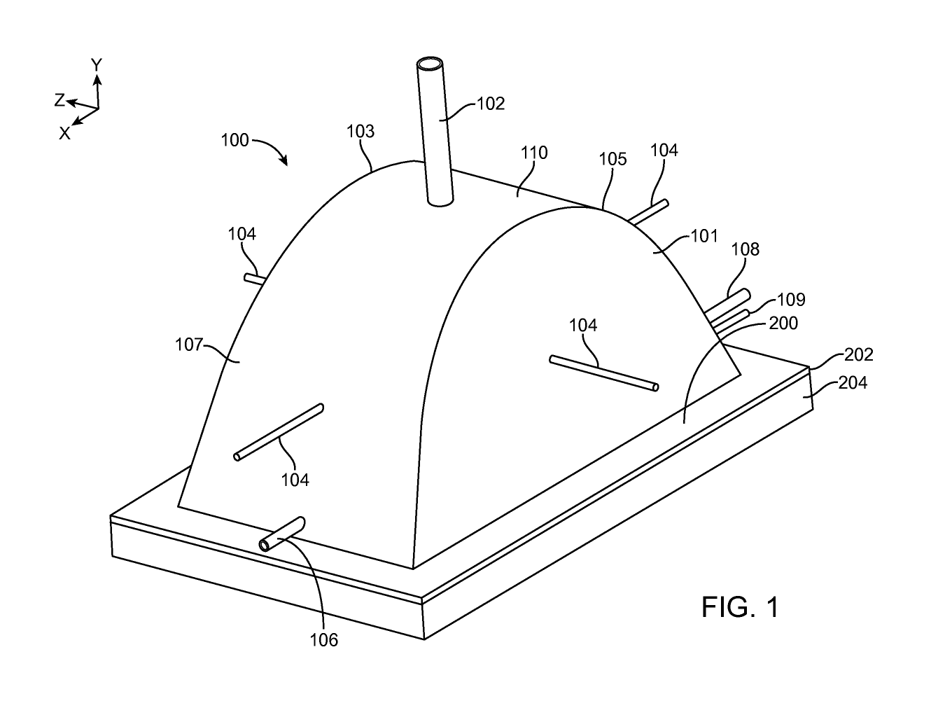

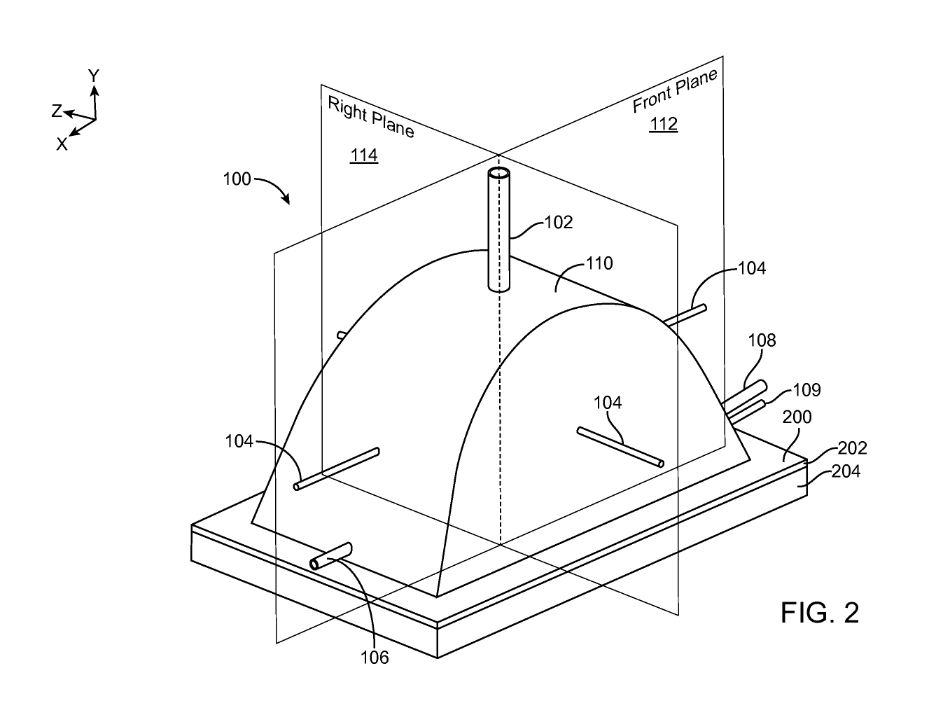

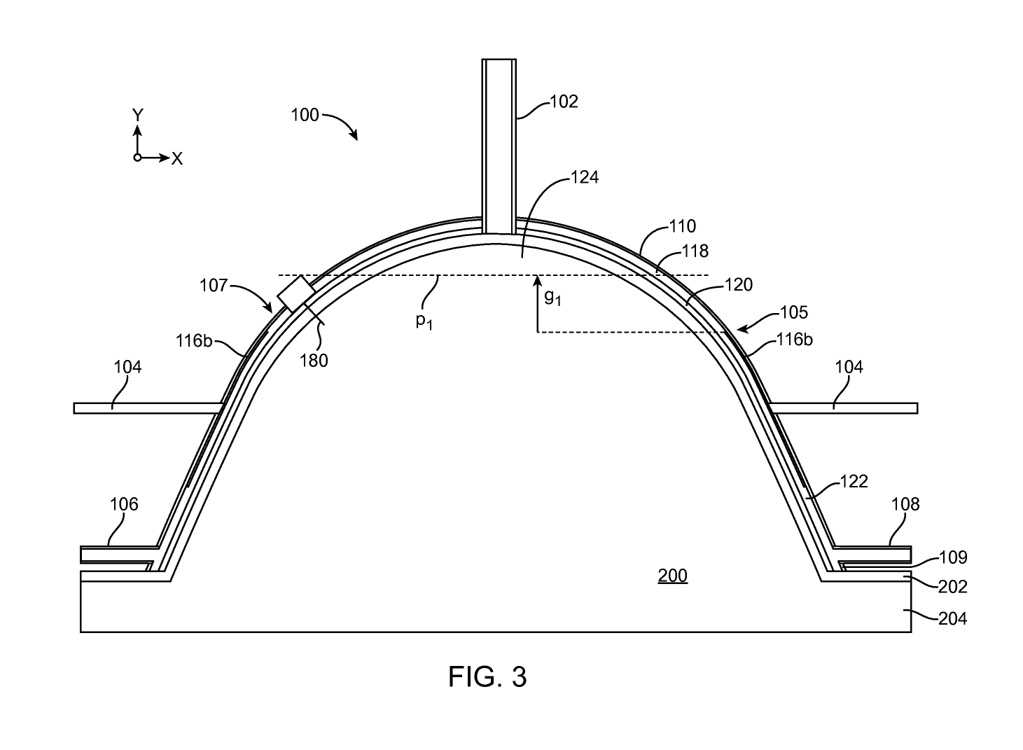

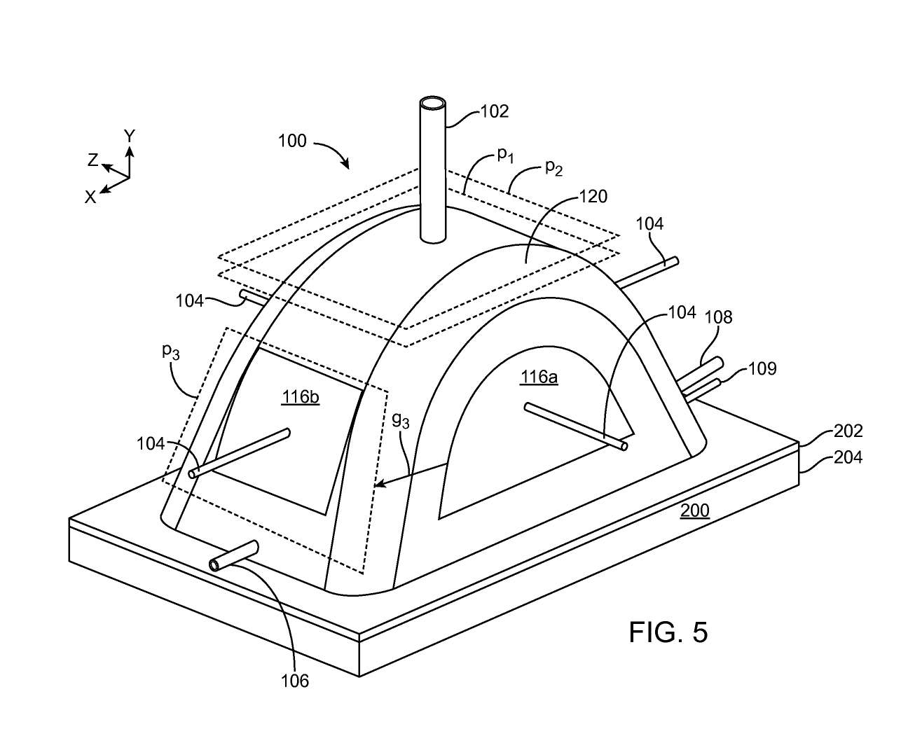

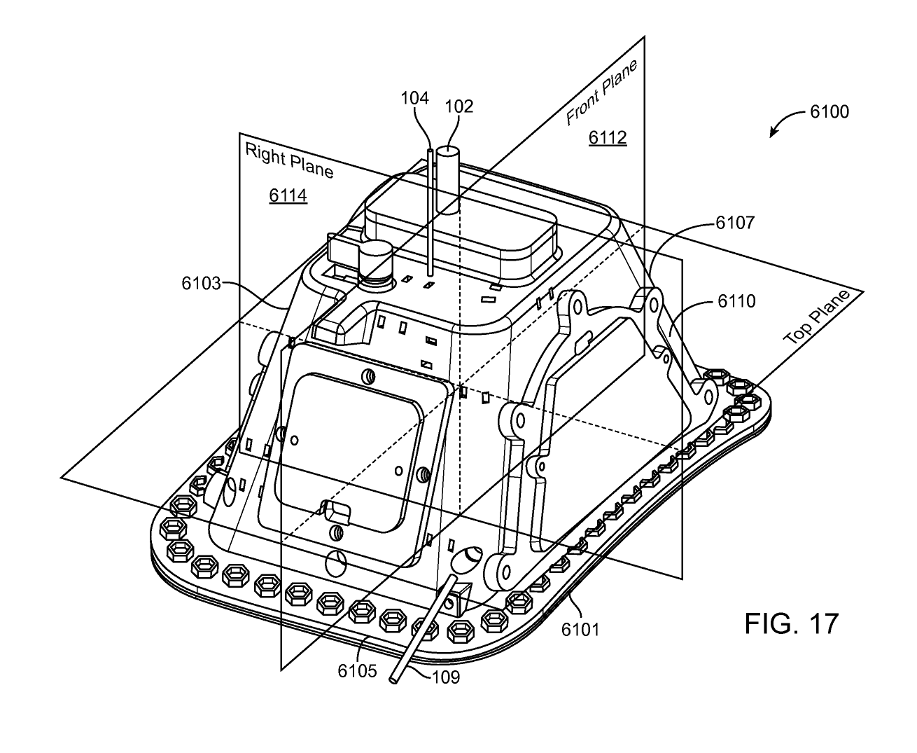

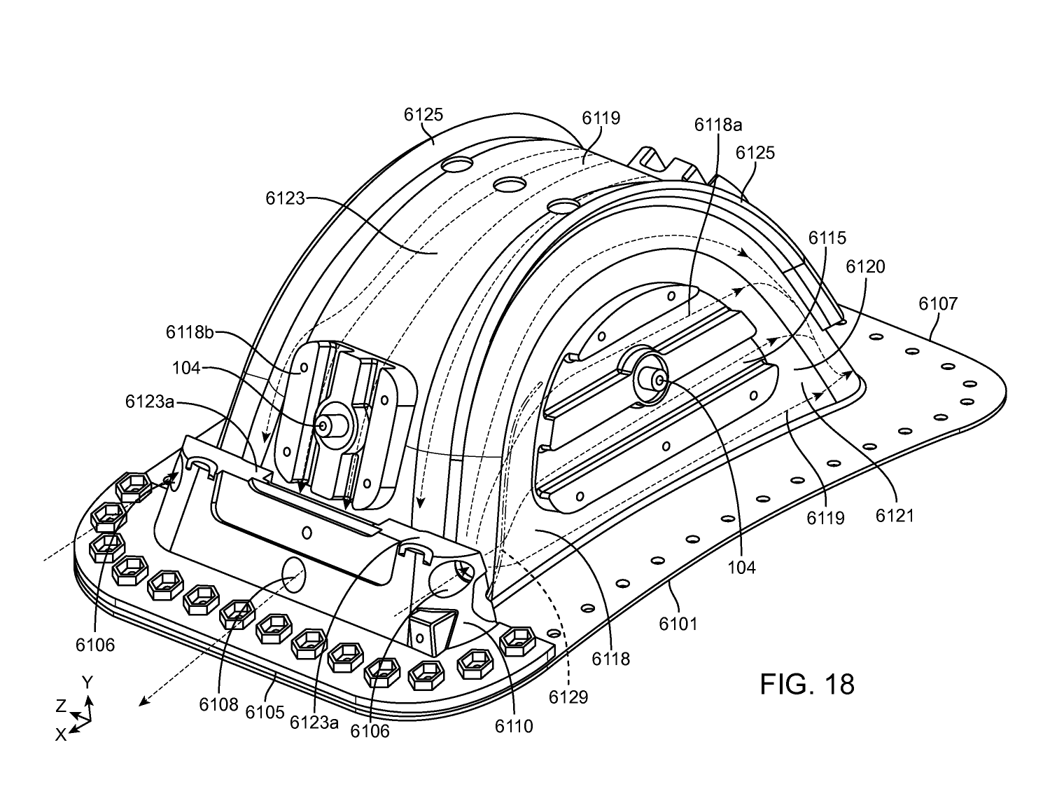

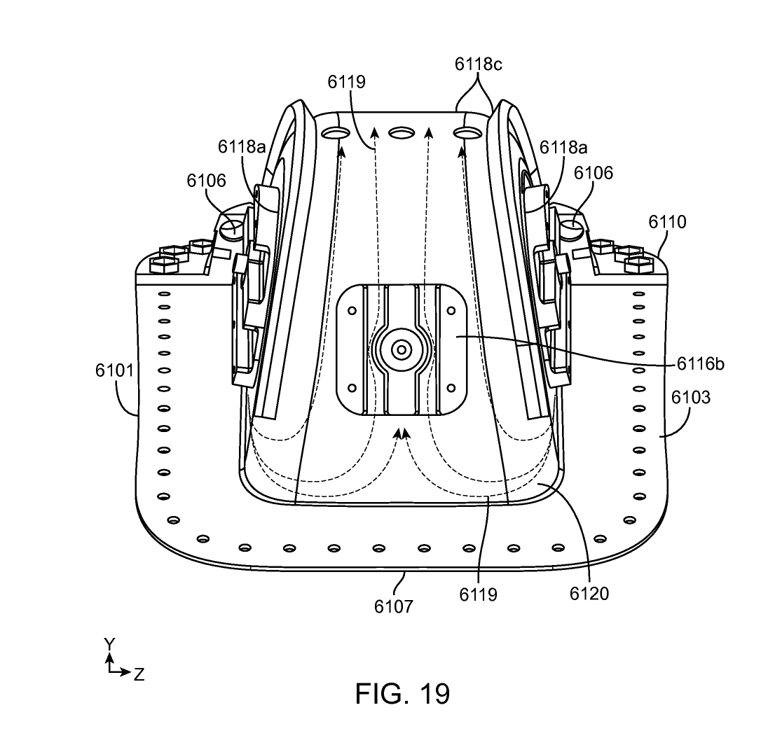

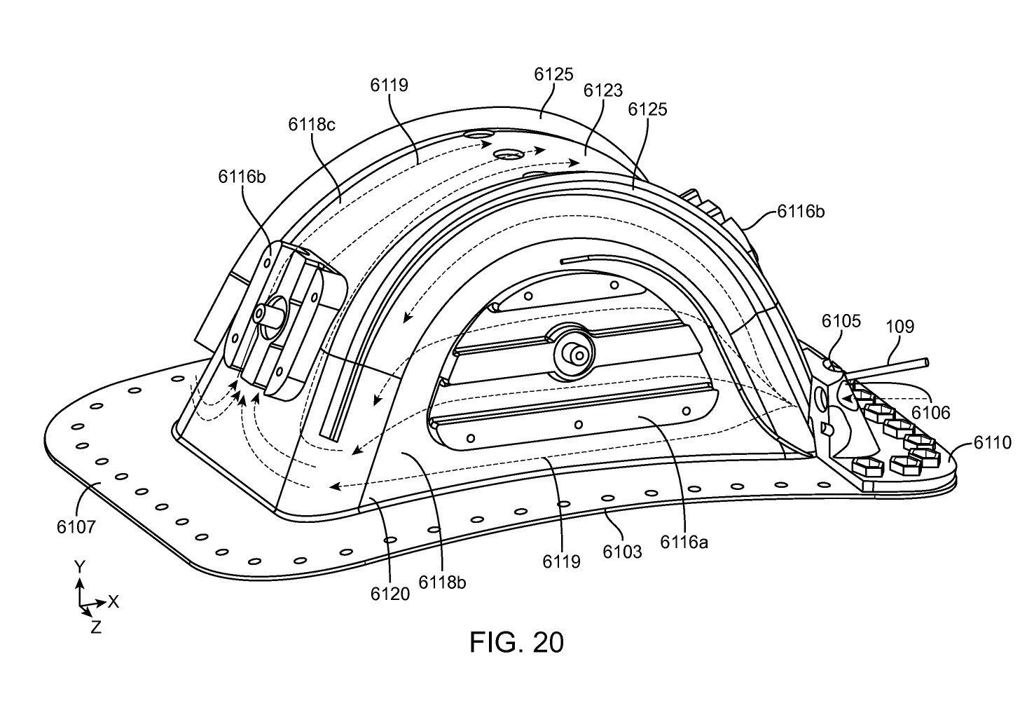

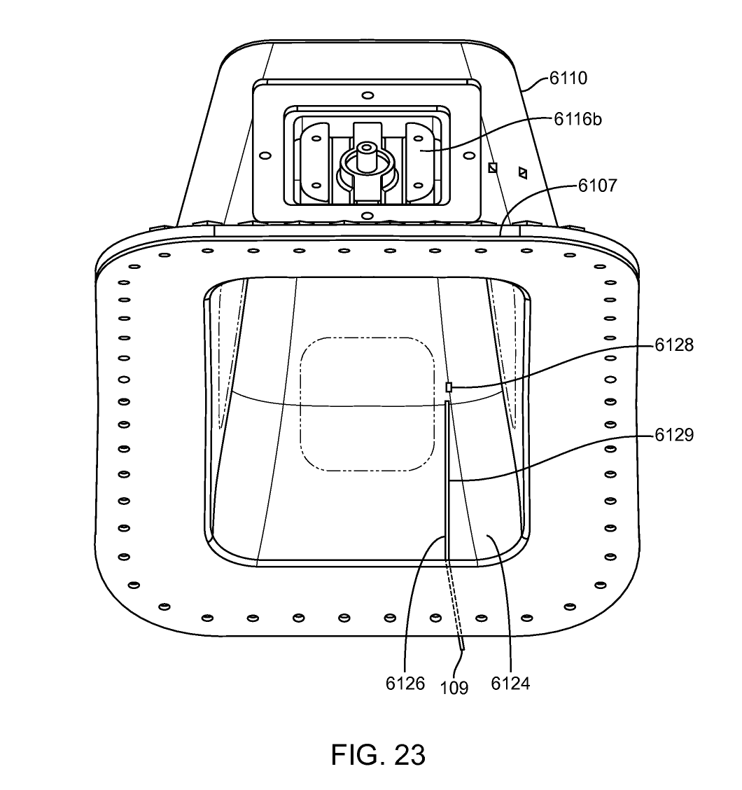

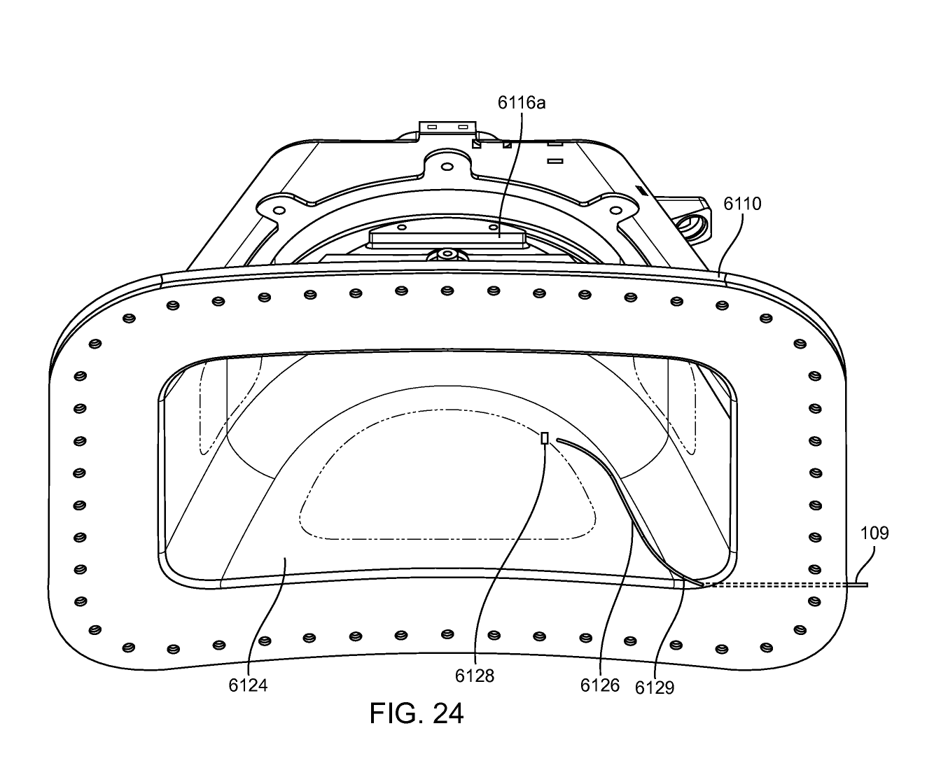

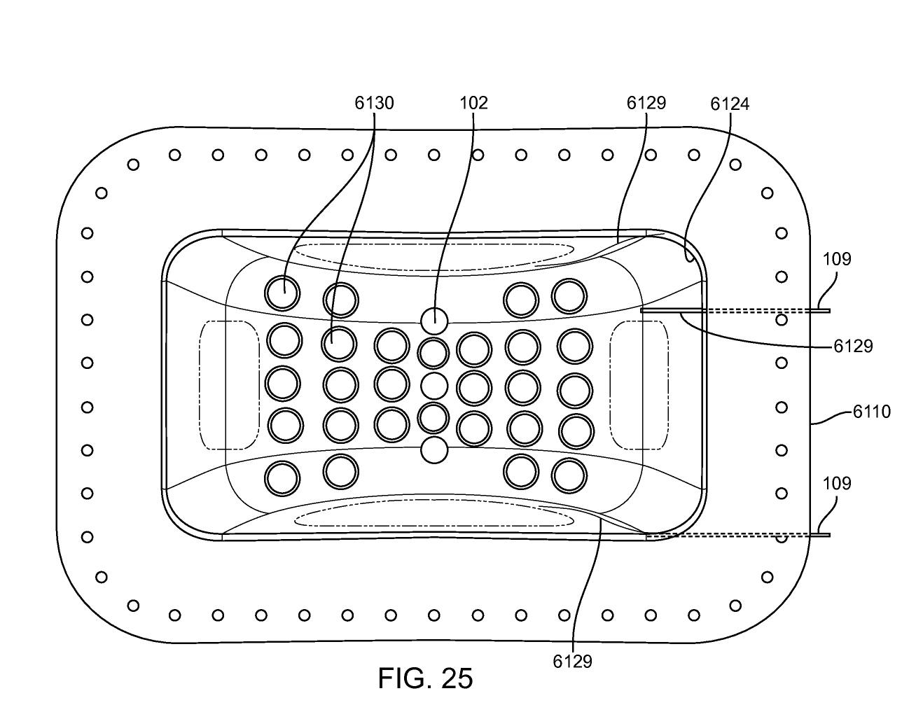

FIGS. 1-5 illustrate an applicator 100 in accordance with aspects of the invention. The applicator 100 is illustrated placed on human layers of tissue 200 (including layers of dermis 202 and fat 204). The applicator 100 comprises an outer housing 110, a vacuum input 102, electrical source inputs or electrode wires 104, a coolant input 106 and a coolant output 108. The applicator 100 may also comprise a sensor output or bus 109 coupled to one or more sensors contained within the housing 110, for example as shown in FIGS. 17-25. The applicator 100 has an arch-shaped, or arced housing 110.

Within the housing 110, a cooling plate 120 forms a vacuum chamber 124 configured to receive the tissue layers 200. The housing 110 and cooling plate 120 may be clear to aid in visualization and ensure desired acquisition of tissue into the vacuum chamber 124. According to some variations, the cooling plate may be constructed of any thermally conductive material or a dielectric material, for example, a ceramic or plastic. The vacuum input 102 provides a vacuum from a vacuum source to the vacuum chamber 124 to draw tissue 200 into the chamber 124. Electrodes 116a are attached within and to sides 101, 103 of the housing 110 and form a first pair of generally opposing electrodes having an arched and curved shape similar to that of the front plane 112 of the applicator 100 and following the curved shape of the cooling plate structure 120. The electrodes in this and other iterations described herein, may be shaped to conform to the walls of the chamber where the tissue is positioned.

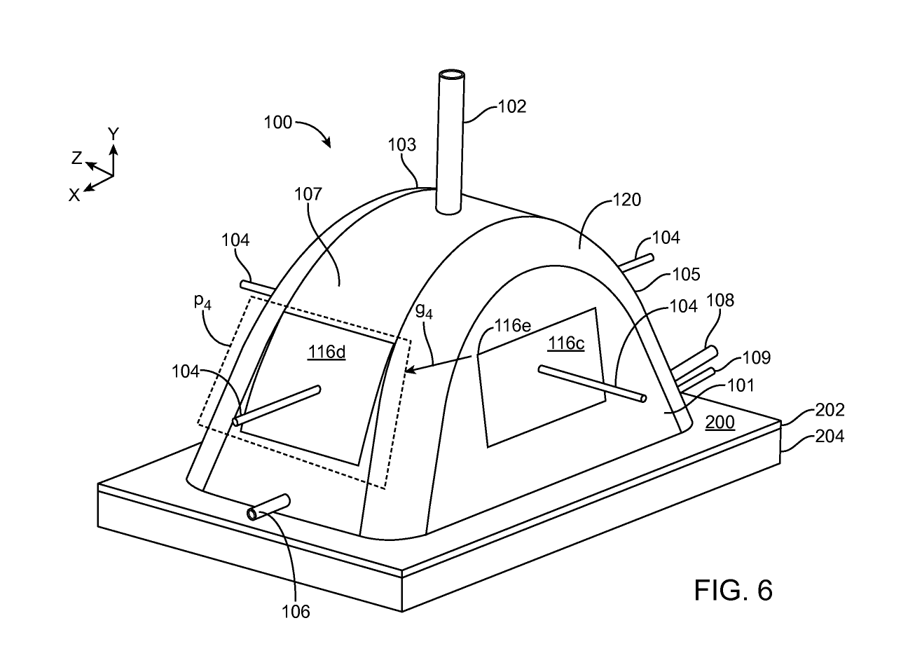

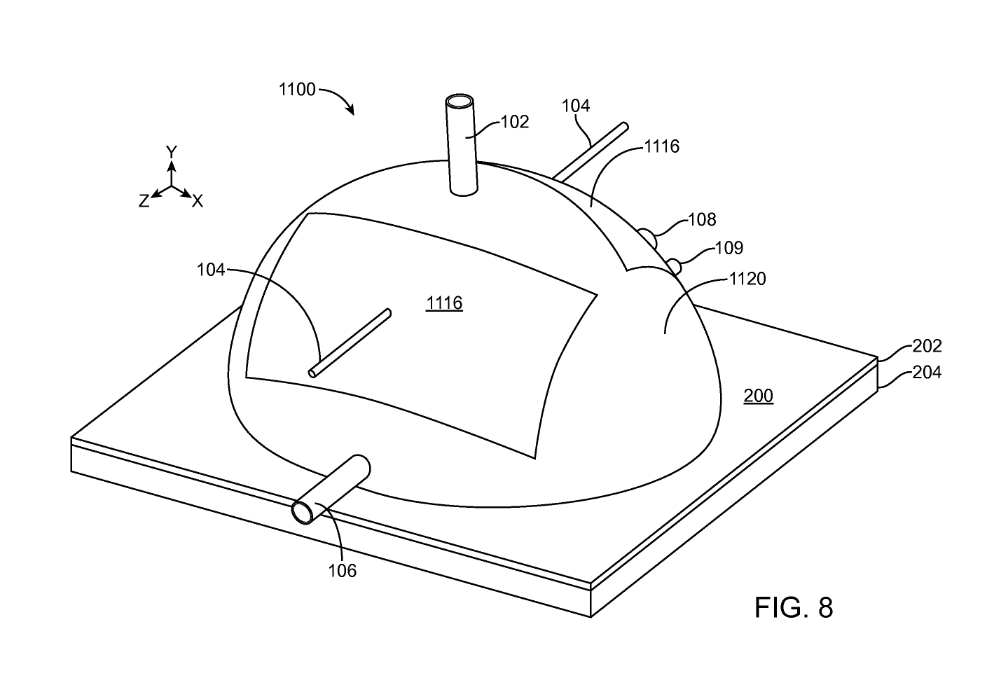

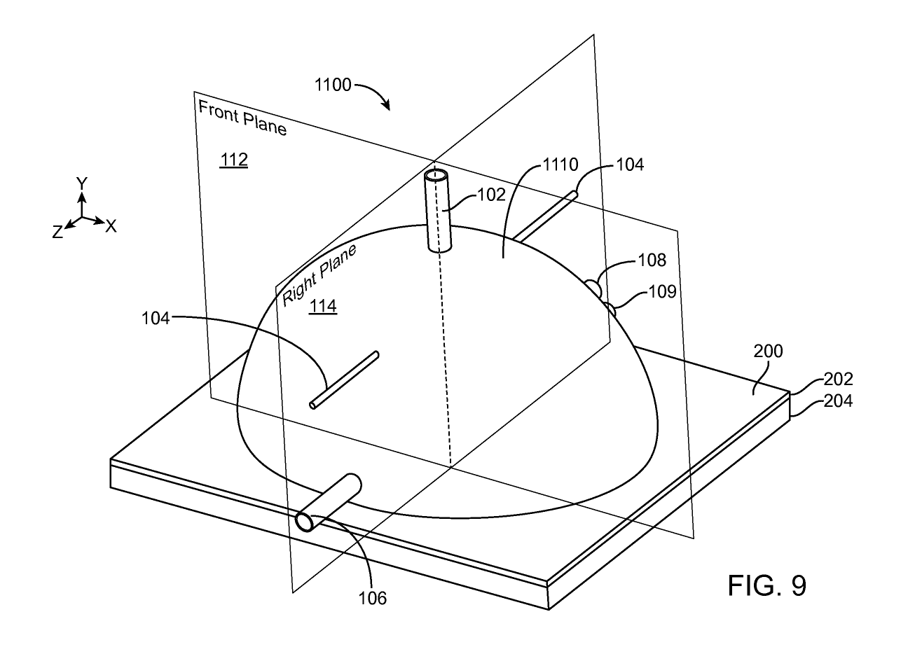

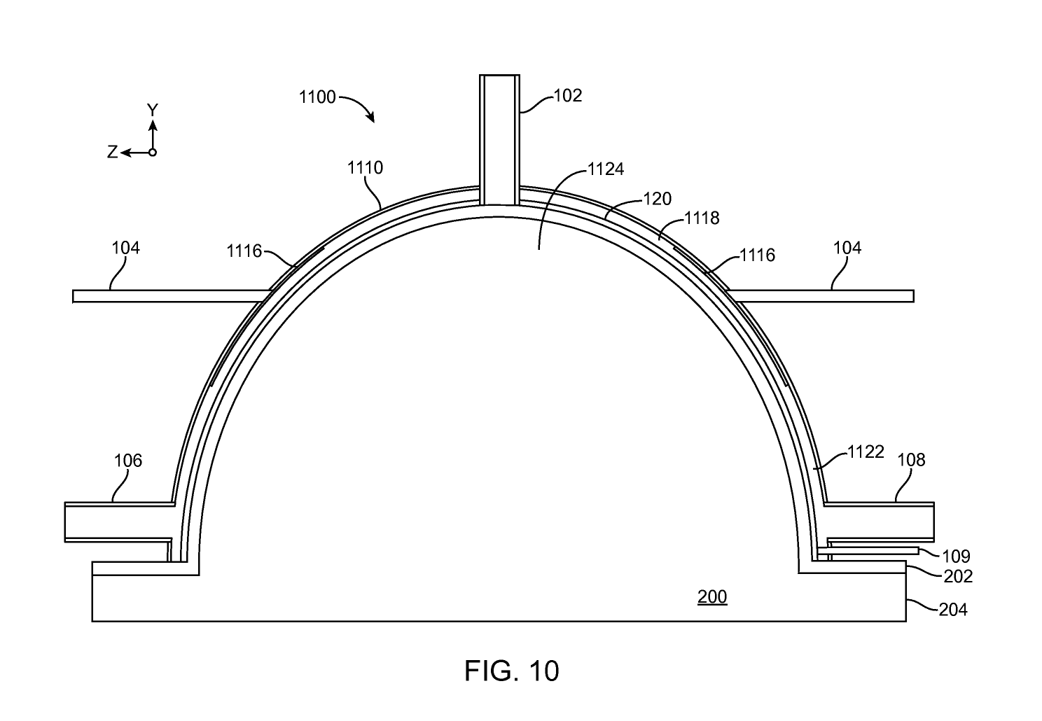

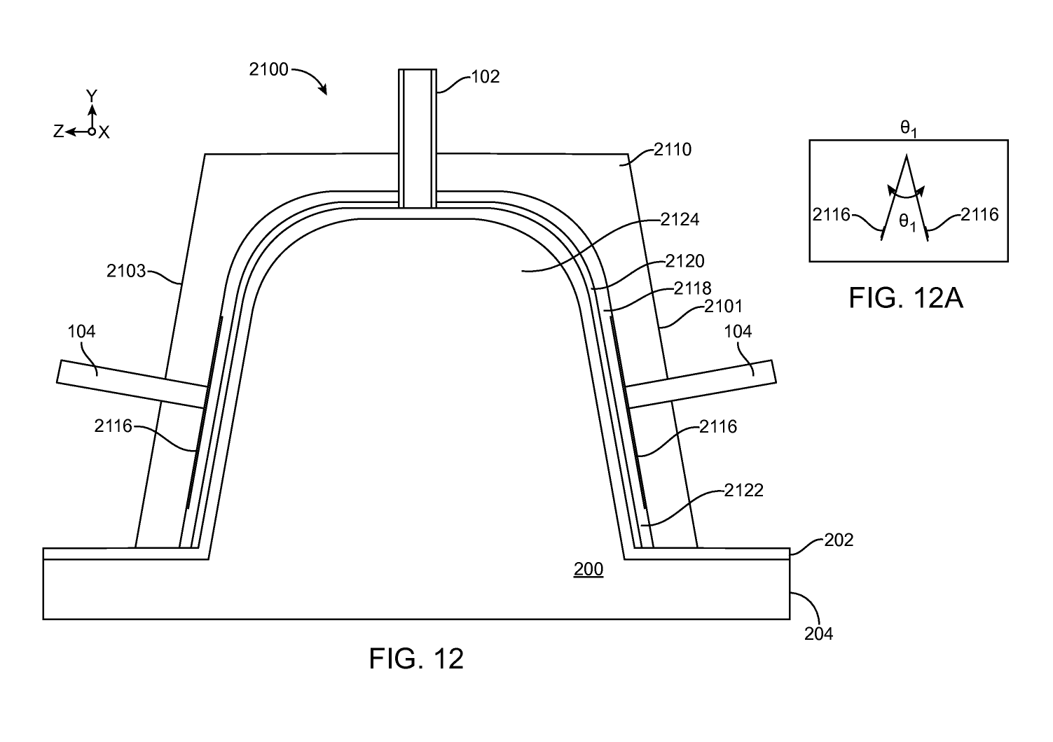

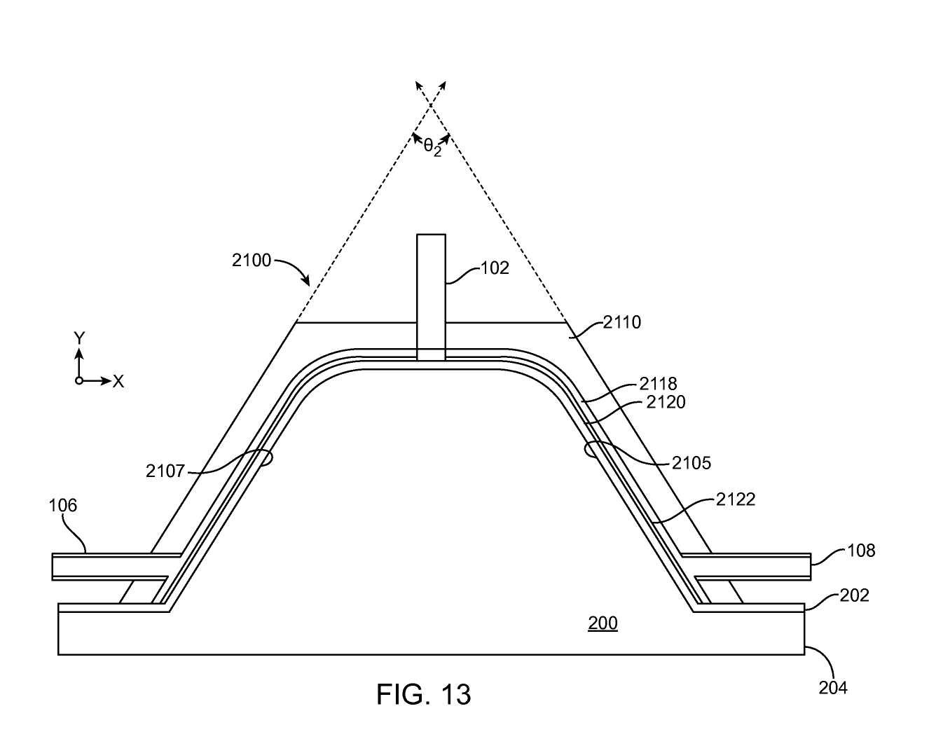

Electrode pairs 116a are illustrated as being generally parallel with respect to each other. Electrodes 116b are attached within and to ends 105, 107 of the housing 110 and form a second pair of generally opposing electrodes shaped to generally fit over the curved geometry of the cooling plate structure 120. The electrode pair 116a are substantially parallel or sufficiently parallel so as to provide a desired amount of energy to the fat tissue. Edges of the electrode pair 116b are spaced by a gap g1, from the edges of the housing or from a plane P1 on the inner walls of the vacuum chamber where tissue in the applicator would bend to a parallel or near parallel orientation with respect to the direction electrical field between electrode pairs to prevent overheating of skin. Edges of the electrode pair 116a are spaced by a gap g3, from the edges of the housing or from a plane P3 on the inner walls of the vacuum chamber where tissue in the applicator would bend to a parallel or near parallel orientation with respect to the direction electrical field between electrode pairs to prevent overheating of skin. Electrode pair 116a is similarly spaced, for example, by a gap g2 from the edges of the housing or from a plane P2 on the inner walls of the vacuum chamber where tissue in the applicator would bend to a parallel or near parallel orientation with respect to the direction electrical field between electrode pairs to prevent overheating of skin. The electrode pairs of FIGS. 6-27 herein may also have similar gaps whether with respect to housing edges on which they are situated or on or adjacent cooling plates where they may be situated.

The electrodes 116a, 116b are energized through wires 104. The housing 110 and electrodes 116a 116b, are spaced from the cooling structure 120 forming a coolant chamber 118 having a flow path from the inlet 106 to the outlet 108. The flow of coolant 122 through the cooling chamber 118 serves to protect the outer layers of the skin while fat is being heated. The shape of the vacuum chamber 124 in combination with the location of the electrodes are configured to provide desired tissue acquisition and contact with the cooling plate 120, as well as to deliver energy at a desired level to tissue and avoid producing hotspots in the tissue.

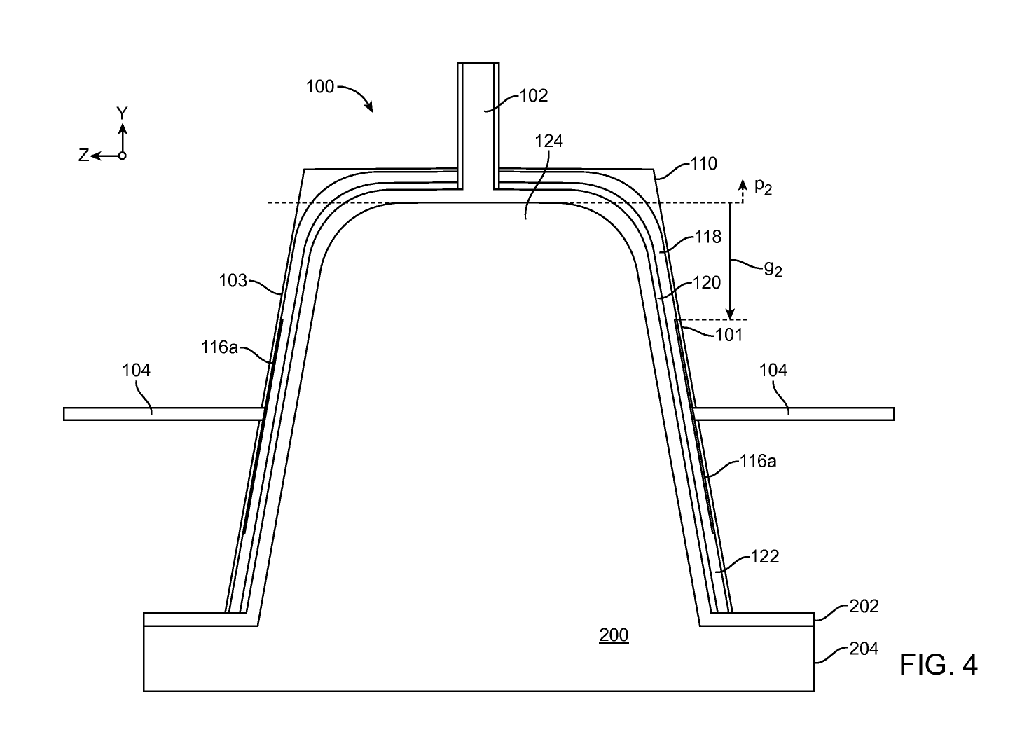

Sensors may be used with any of the applicators described herein. Sensors shown in any particular applicator or otherwise described herein may be used in any applicator according to the invention including but not limited to applicators illustrated herein. FIG. 4 schematically illustrates a needle temperature sensor 180 that may be incorporated into the applicator and may be positioned with its tip within a therapeutic or target zone to get a reading on the temperature in the zone. The temperature readings may be used as a feedback mechanism to control the energy delivery. The needle sensor may be actuated (e.g. pushed) and inserted into through the skin into a treatment zone after tissue has been drawn into the acquisition chamber.

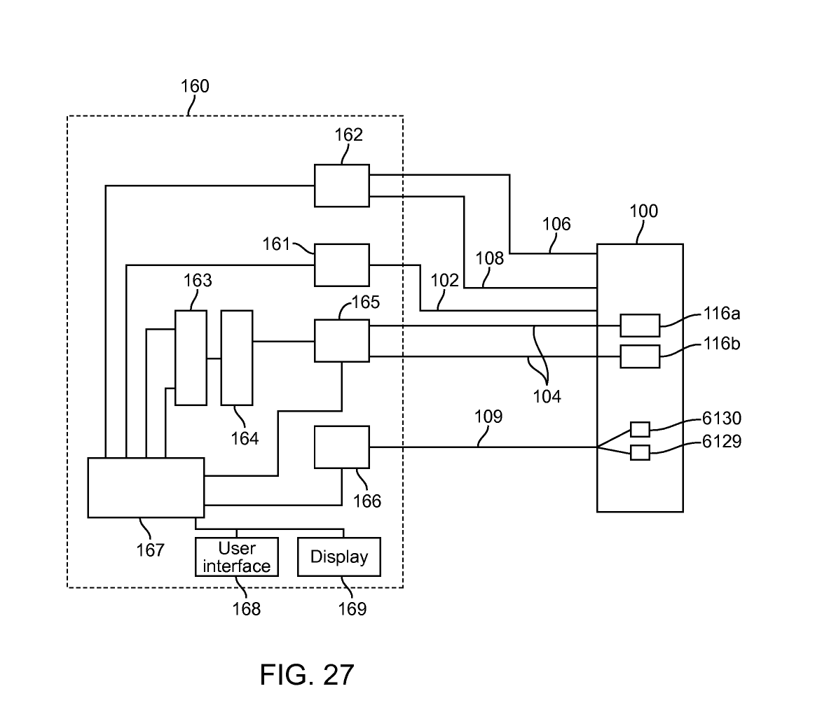

A system in accordance with the invention is illustrated in FIG. 27. The applicator 100 is configured to be coupled to a control console 160 that includes a vacuum source 161, chiller/heater 162, function generator 163, amplifier 164, switch 165, sensor inputs 166 and a controller 167. The controller 167 may control the generator 163, amplifier 164 and the switch 165 to provide desired input through the electrode wires 104 to electrodes 116a, 116b according to a treatment protocol. The treatment protocol may include, for example, delivering different frequencies, power levels, or treatment durations at each electrode pair. The treatment protocol may also comprise providing different coolant temperatures or flow rates to electively cool tissue during treatment. The protocol may also provide separate coolant delivery to different coolant chambers and varying the coolant temperature or flow rate to each of the different chambers to thereby control the volumetric size and/or shape of the therapeutic zone.

According to variations, both the electrical parameters to the electrodes and the coolant parameters may be used to control the therapeutic zone size and shape. The vacuum source 161 is coupled to the vacuum input 102 of the applicator 100 and may or may not be controlled by the controller 167 to provide vacuum input to the vacuum chamber 124 by way of the vacuum input 102. The control console 160 also includes a chiller/heater 162 to circulate a coolant 120 to and from the applicator 100 through coolant input 106 and output 108 and may or may not be controlled by the controller 167. If sensors 6129, 6130 180, or other sensors are used to provide feedback from the applicator 100, they may be output to the controller 167 through one or more sensor outputs or bus 109.

The applicator 100 may also include one or more connectors configured to couple it with the control unit and to provide feedback to the control unit regarding connection status or other information concerning the applicator 100 the tissue engaged by the applicator 100. The controller may also comprise a display 168 and a user interface 169.

FIG. 6 illustrates an applicator 100 constructed similarly to applicator 100 in FIGS. 1-5. Electrode pairs 116c and 116d are rectangular shaped with electrode pair 116c on the sides 101, 103 of the applicator 300 being larger in area than electrode pair 116d on the ends 105, 107 of the applicator 300 corresponding to the relative dimensions of the tissue contacting walls inside the vacuum chamber 124. Edge 116e of the electrode pair 116c are spaced by a gap g4, from the edges of the housing or from a plane P4 on the inner walls of the vacuum chamber where tissue in the applicator would bend to a parallel or near parallel orientation with respect to the direction electrical field between electrode pairs to prevent overheating of skin. Other electrode gaps as well may be provided to reduce hot spots. The applicator may also be coupled to a control console 160 as described with respect to FIGS. 1-5.

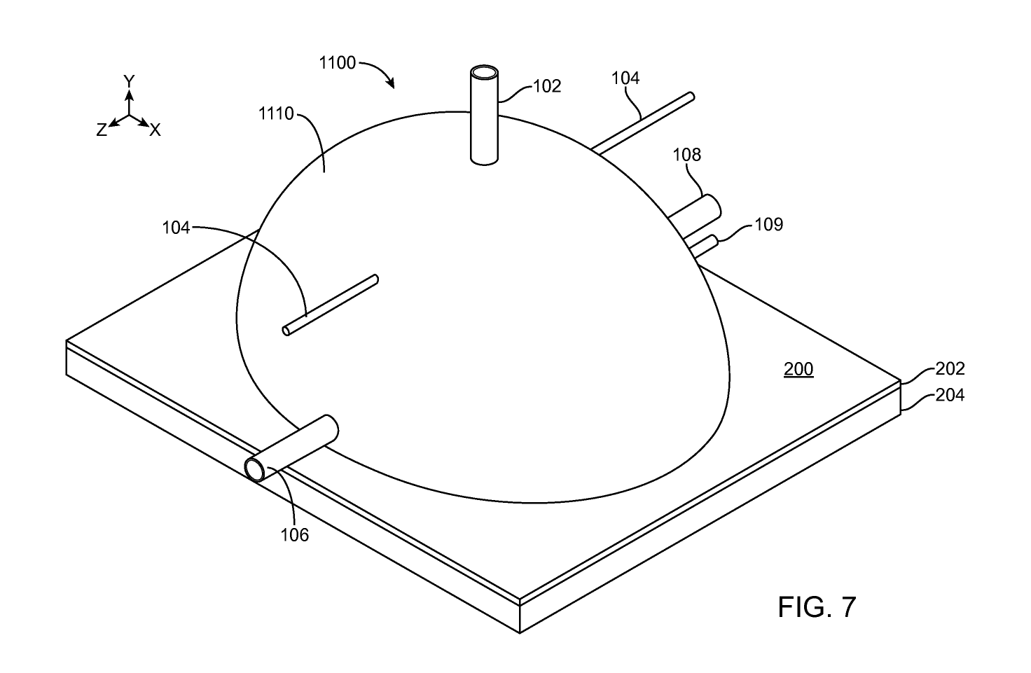

FIGS. 7-10 illustrate an applicator 1100 in accordance with aspects of the invention. The applicator 1100 is illustrated placed on human layers of tissue 200 (including layers of dermis 202 and fat 204). Similar to applicators 100 and 300, the applicator 1100 may be configured to be coupled to a control module 160 for example, as shown in FIG. 27. The applicator 1100 may also include one or more connectors configured to couple it with the control console 160 and to provide feedback to the control console 160 regarding connection status or other information concerning the applicator 1100 or the tissue engaged by the applicator 1100.

The applicator 1100 comprises an outer housing 1110, a vacuum input 102, electrical source inputs or electrode wires 104, a coolant input 106 and a coolant output 108. The applicator 1100 has an elliptical dome shaped housing 1110 in this iteration. Within the housing 1110, a cooling structure or a cooling plate 1120 forms a vacuum chamber 1124 configured to receive the tissue layers 200. The chamber 1124 may substantially follow the dome shape of the housing 1110. The cooling plate 1120 may be constructed of any thermally conductive material or a dielectric material, for example, a plastic or ceramic. The vacuum input 102 provides a vacuum from a vacuum source to the vacuum chamber 1124 to draw tissue 200 into the chamber 1124.

A single pair of electrodes 1116 are attached within the housing 1110 and follow the curved elliptical dome shape of the cooling plate 1120 and form a pair of generally opposing electrodes. The electrodes 1116 are energized through wires 104. The housing 1110 and electrodes 1116 are spaced from the cooling structure 1120 forming a coolant chamber 1118 having a flow path from the inlet 106 to the outlet 108. The flow of coolant 1122 through the cooling chamber 1118 serves to protect the outer layers while the fat is being heated.

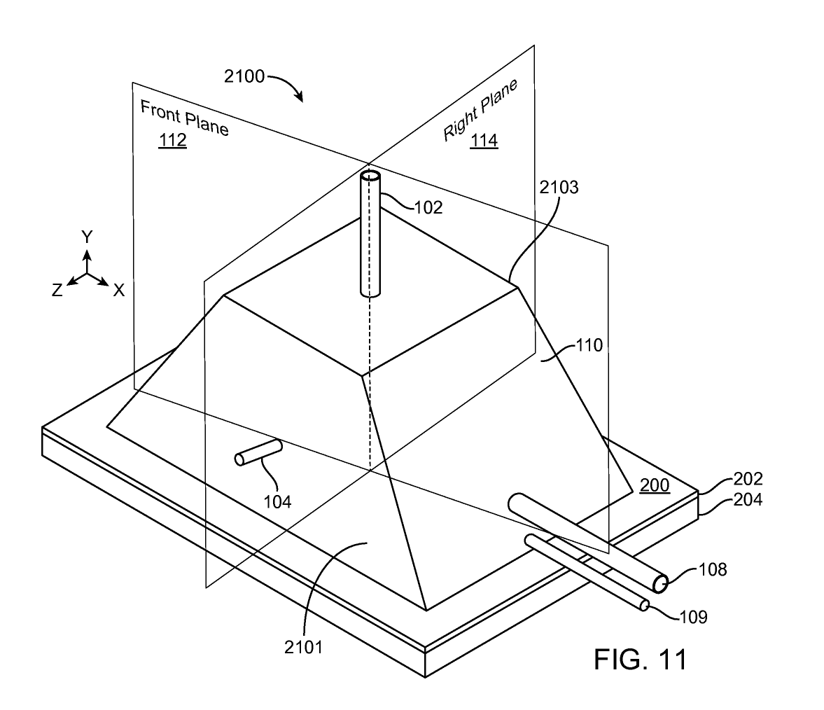

FIGS. 11-13 illustrate an applicator 2100 in accordance with aspects of the invention. The applicator 2100 is illustrated placed on human layers of tissue 200 (including layers of dermis 202 and fat 204). Similar to applicators 100, 300, 1100, the applicator 2100 may be configured to be coupled to a control console 160 for example, as shown in FIG. 27. The applicator 2100 may also include one or more connectors configured to couple it with the control unit and to provide feedback to the control console 160 regarding connection status or other information concerning the applicator 2100 the tissue engaged by the applicator 2100.

The applicator 2100 comprises an outer housing 2110, a vacuum input 102, electrical source inputs or electrode wires 104, a coolant input 106 and a coolant output 108. The applicator 2100 has a trapezoidal shaped housing 2110 in this iteration.

Within the housing 2110, a cooling plate 2120 forms a vacuum chamber 2124 configured to receive the tissue layers 200. The cooling plate 2120 may be constructed of any thermally conductive material or a dielectric material, for example, a plastic or ceramic. The vacuum input 102 provides a vacuum from a vacuum source to the vacuum chamber 2124 to draw tissue 200 into the chamber 2124. Electrodes 2116 are attached within and to sides 2101, 2103 of the housing 2110 and form a pair of generally opposing electrodes having an orientation or angle θ1 of 45° or less with respect to each other. Inner sides 2105, 2107 of chamber 2124 are configured to assist in providing a desirable and adequate draw of tissue into the chamber 2124 as well as to provide increased tissue surface area contact with the cooling plate 2124. Accordingly in some embodiments, the inner sides 2105, 2107 of chamber 2124 form an angle θ2 with respect to each other that is greater than the angle θ1 the electrodes 2116 form with respect to each other. In some embodiments the angle θ2 that the inner side walls 2105, 2107 form with respect to each other is about 45° or more. The electrodes 2116 are energized through wires 104. The housing 2110 and electrodes 2116 are spaced from the cooling structure 2120 forming a coolant chamber 2118 having a flow path from the inlet 106 to the outlet 108. The flow of coolant 2122 through the cooling chamber 2118 serves to protect the outer layers of the skin while fat is being heated.

In embodiments of the invention the electrodes forming the capacitor plates may be adjusted prior to initiating treatment and held fixed during treatment. Alternatively, the electrodes may be adjusted during the treatment.

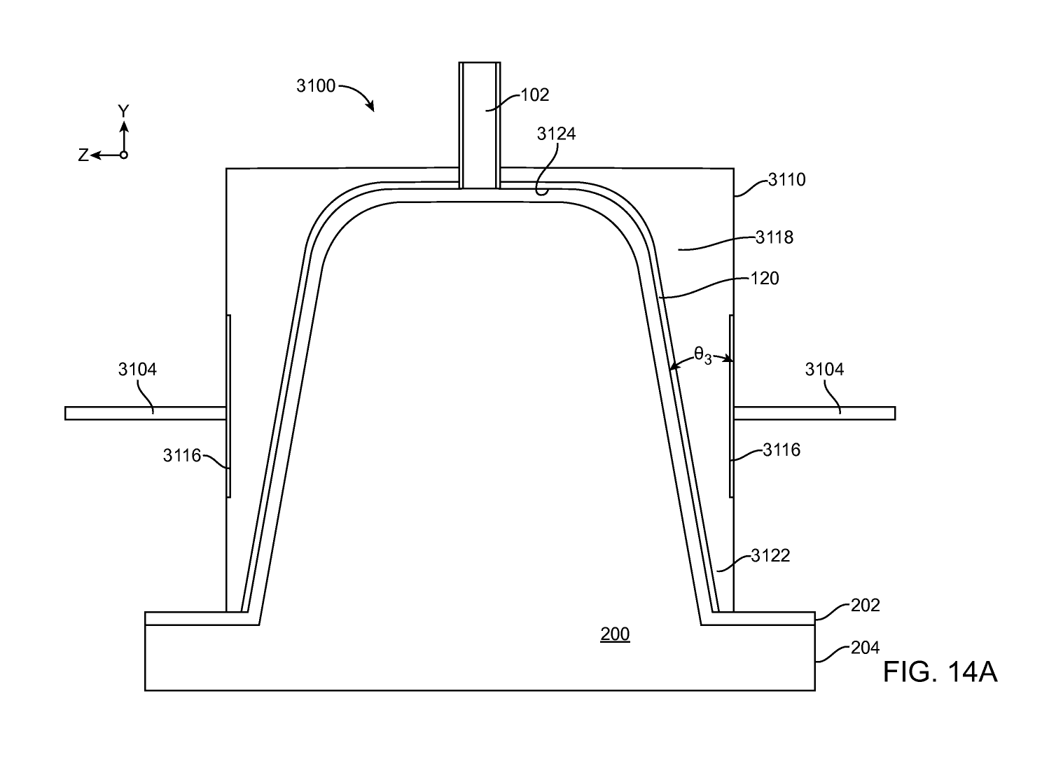

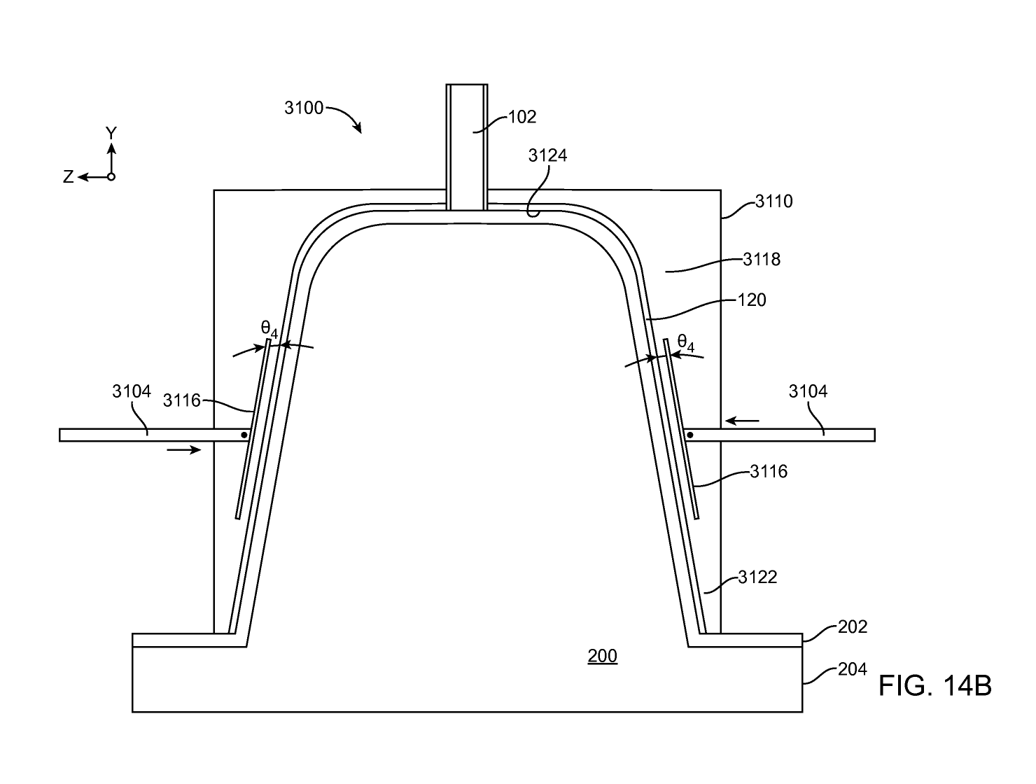

FIG. 14A illustrates an alternative construction of an applicator 3100. In this instance, the electrodes 3100 form a parallel oriented pair of electrodes in a first position within the housing 3100. It is believed that a parallel orientation of electrodes provides a generally uniform distribution of energy to fat tissue between the electrodes. The electrodes 3116 are shown in a first configuration where they form an angle θ3 greater than 0, with respect to the inner walls 3101, 3103 of the vacuum chamber 3124, and thus with respect to the tissue interface with the walls 3101, 3103. Thus while the electrodes are parallel, the tissue acquisition chamber 3124 has a wider opening at the bottom to assist in drawing tissue deeper into the chamber 3124 and provide contact with the cooling plate 3120 at the top of the vacuum chamber 3124.

The electrodes are movable or pivotable, e.g., with a movable electrode connector 3104, such as shown in FIG. 14B. As shown in FIG. 14B electrodes 3116 are shown in a second configuration with an angle θ4 is formed between the walls 3101, 3103 and respective adjacent electrodes 3116, According to variations, angle θ4 is less than 03. According to variations, the electrode plate 3116 is parallel with adjacent inner wall 3101 or 3103 of the vacuum chamber 3124.

Similar to applicators 100, 300, 1100, 2100, the applicator 3100 may be configured to be coupled to a control console 160 for example, as shown in FIG. 27. The applicator 3100 may also include one or more connectors configured to couple it with the control console 160 and to provide feedback to the control console 160 regarding connection status or other information concerning the applicator 3100 or the tissue engaged by the applicator 3100.

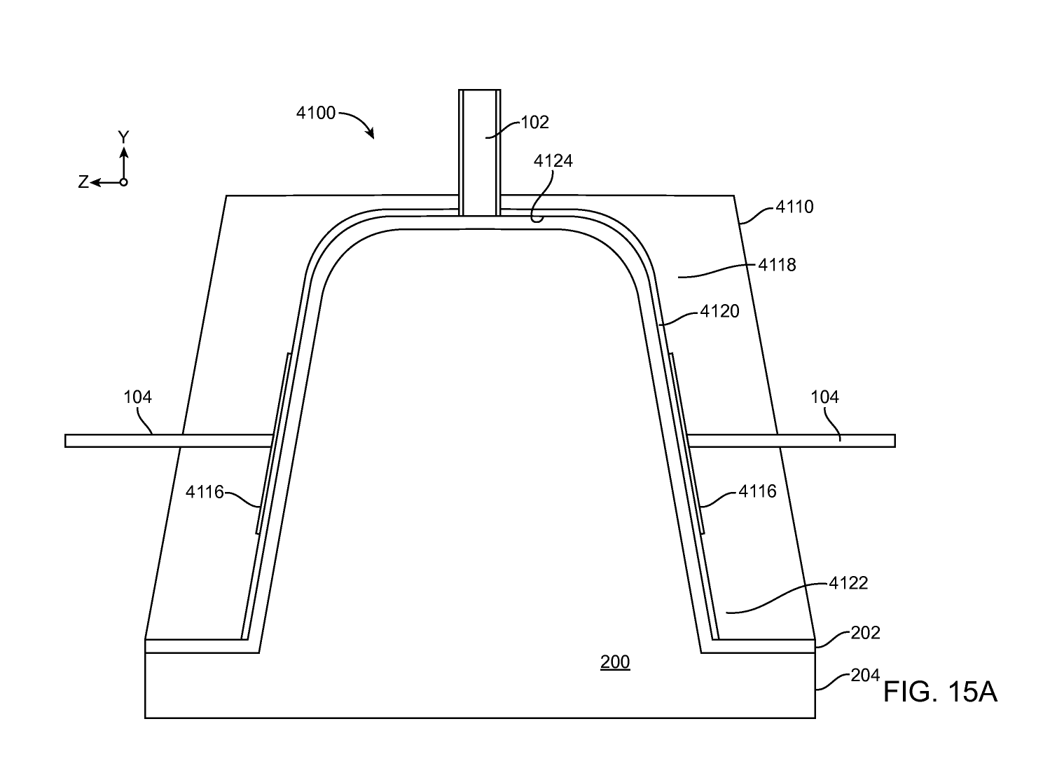

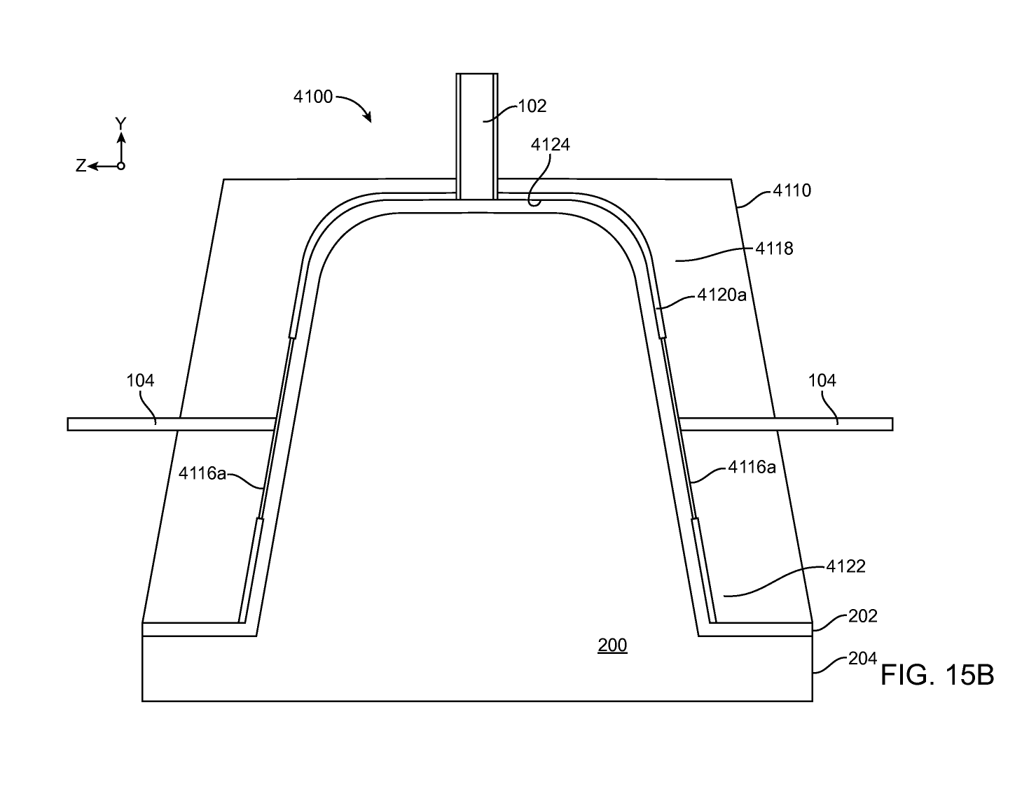

FIG. 15A illustrates an alternative construction of an applicator 4100. In this instance, the electrodes 4116 are directly coupled to the outside of the cooling plate 4120. The housing 4110 and cooling plate 4120 form a cooling chamber 4118. The cooling plate 4120 forms a vacuum chamber 4124. The coolant 4122 circulates within the cooling chamber 4118 and around both the cooling plate 4120 and electrodes 4116. Similar to applicators 100, 300, 1100, 2100, 3100 the applicator 4100 may be configured to be coupled to a control console 160 for example, as shown in FIG. 27. The applicator 4100 may also include one or more connectors configured to couple it with the control console 160 and to provide feedback to the control console 160 regarding connection status or other information concerning the applicator 4100 or the tissue engaged by the applicator 4100.

FIG. 15B illustrates an alternative construction of an applicator 4100. In this instance, the electrodes 4116a are generally contiguous with the inner surface of the vacuum chamber. The electrodes are sealingly positioned within a cutout of the cooling plate 4120a. The housing 4110 forms a cooling chamber 4118 with and external to the electrodes 4116a and cooling plate 4120a. The cooling plate 4120 and electrodes 4116a form a vacuum chamber 4124. The coolant 4122 circulates within the cooling chamber 4118 and around both the cooling plate 4120 and electrodes 4116a. The electrodes may include coolant channels as described herein with reference to FIGS. 17-25. Similar to applicators 100, 300, 1100, 2100, 3100 the applicator 4100 may be configured to be coupled to a control console 160 for example, as shown in FIG. 27. The applicator 4100 may also include one or more connectors configured to couple it with the control console 160 and to provide feedback to the control console 160 regarding connection status or other.

In embodiments of the invention, heating patterns may be selected by a user or a controller/program. In variations, multiple electrodes may be positioned on either side of the applicator chamber, which electrodes may be fixed or adjustable. The electrodes may selectively deliver varying amounts of energy to therapeutic zones in the tissue. According to variations, the electrode parameters may be selected to control the volumetric size and shape of the therapeutic zone to thereby provide contouring of the tissue. Some iterations utilize multiple sets of electrodes which may be angled independently and spaced for optimal heating. Such electrodes may have different sizes or shapes. Alternatively or in additions, coolant parameters such as temperature and flow rate may be used to control the volumetric shape and size of the therapeutic zone and to thereby provide contouring of the tissue. Multiple flow chambers with different coolant temperatures or flow rates may be used for controlling the therapeutic zone or contouring.

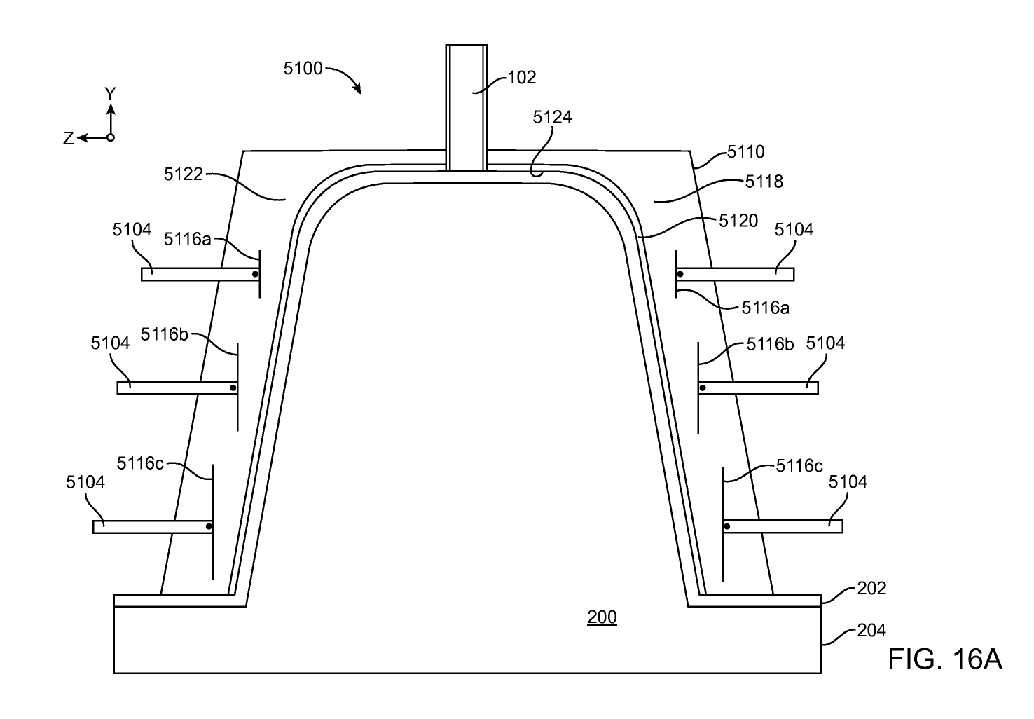

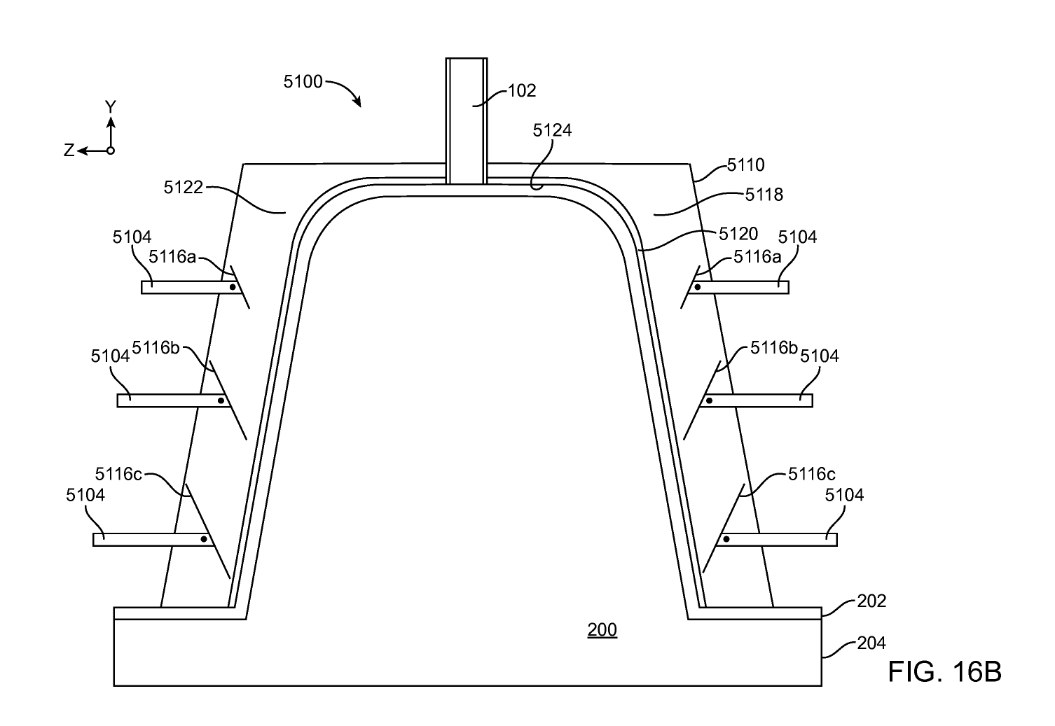

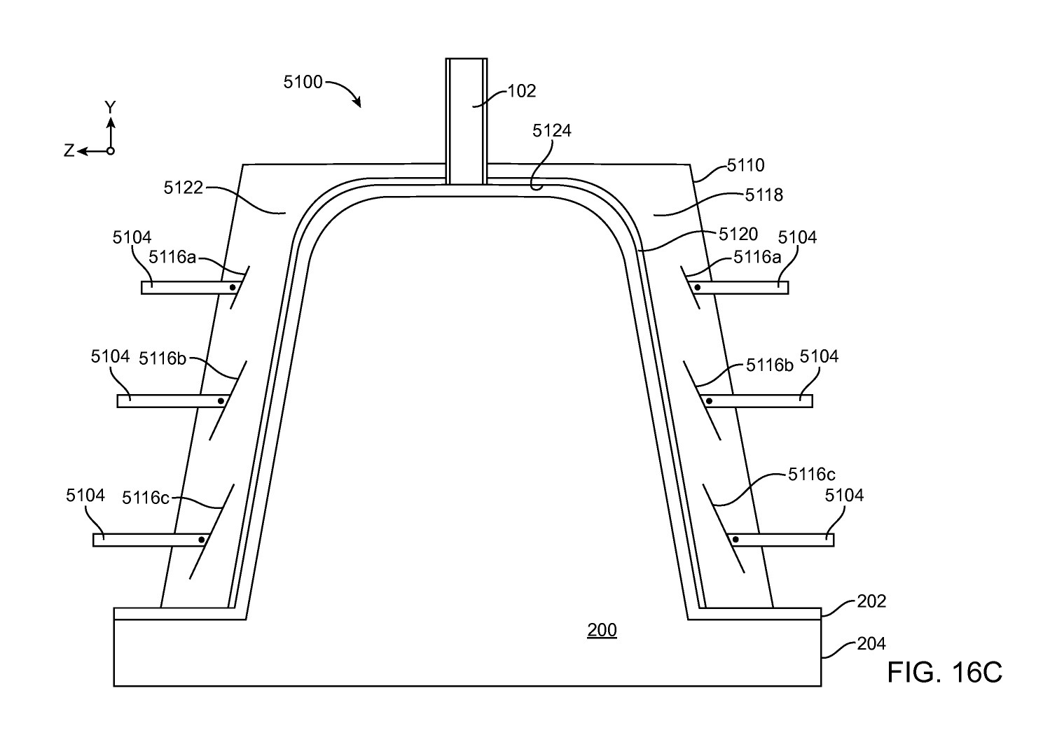

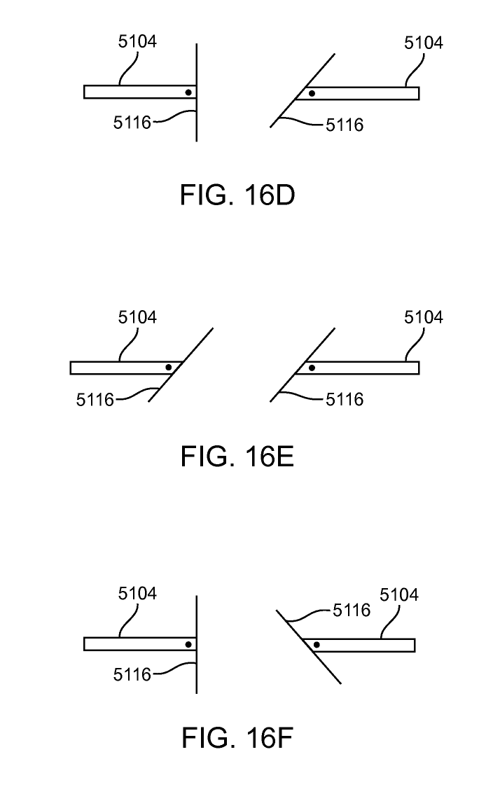

FIGS. 16A-F illustrate an alternative construction of an applicator 5100. In this instance, multiple electrodes 5116a, 5116b, and 5116c form three independent electrode pairs positioned within housing 5110. As illustrated electrodes 5116a, 5116b and 5116c have different dimensions. In this particular illustration, the size of the electrodes are different with 5116a being the smallest and 5116c being the largest. They may also have different surface areas, different lengths, widths or different shapes. Electrode dimension as used herein are used to mean electrode shapes, sizes surface areas, widths, lengths, depths, volumes or different construction that may provide a varied or different energy delivery characteristic to tissue. The housing 5110 and the cooling plate 5120 form a cooling chamber 5118 in which the electrodes are positioned and through which coolant 5120 flows. The electrode may be separated from the cooling chamber as well and may be movable to different configurations or orientations within an isolated chamber within the housing. The cooling plate 5120 forms the vacuum chamber 5124 into which the tissue 200 is drawn. The electrodes 5116a, 5116b, 5116c may be alternatively actuated according to a protocol and may be controlled by a controller such as, for example, as described with respect to control console 160 in FIG. 27. The multiple electrodes 5116a, 5116b, 5116c may be movable or pivotable in the same manner as described with respect to electrodes 3116 in FIGS. 14A and 14B.

FIG. 16A shows multiple electrode pairs 5118a, 5118b, 5118c positioned on each side of the acquisition chamber in a first configuration where the electrodes are generally parallel with respect to each other. FIG. 16B shows multiple electrode pairs 5118a, 5118b, 5118c positioned on each side of the acquisition chamber in a second configuration where the electrodes are closer to each other at the lower ends and have a greater distance between them at their upper ends. Thus, the electric field is stronger at their lower ends and in this configuration may be directed to regions of tissue in the vacuum chamber where there is greater volume. FIG. 16C shows multiple electrode pairs 5118a, 5118b, 5118c positioned on each side of the acquisition chamber in a third configuration where the electrodes are closer to each other at the lower ends and have a greater distance between them at their lower ends. Thus, the electric field is stronger at their lower ends and in this configuration volume. FIG. 16D shows electrodes 5118 of the applicator of FIG. 16A in a fourth configuration. FIG. 16E shows electrodes 5118 of the applicator 5100 of FIG. 16A in a fifth configuration. FIG. 16F shows electrodes 5118 of the applicator 5100 of FIG. 16A in a sixth configuration. Each of the electrode pairs 5116a, 5116b, and 5116c may have different configurations from the other of the electrode pairs and thus may be used to contour the fat in numerous different manners as selected either by a user or a corresponding program or protocol.

FIGS. 17-25 illustrate an applicator 6100 in accordance with aspects of the invention. The applicator 6100 may be placed on human layers of tissue (including layers of dermis and fat). The applicator 6100 comprises an outer housing 6110, a vacuum input 6102, electrical source inputs or electrode wires 6104, a coolant input 6106 and a coolant output 6108. The applicator 6100 has an arch-shaped, or arced housing 6110 in this iteration. Similar to applicators 100, 300, 1100, 2100, 3100, 4100, 5100, applicator 6100 may be configured to be coupled to a control console 160 for example, as shown in FIG. 27. The applicator 6100 may also include one or more connectors configured to couple it with the control console 160 and to provide feedback to the control console 160 regarding connection status or other information concerning the applicator 6100 or the tissue engaged by the applicator 6100.

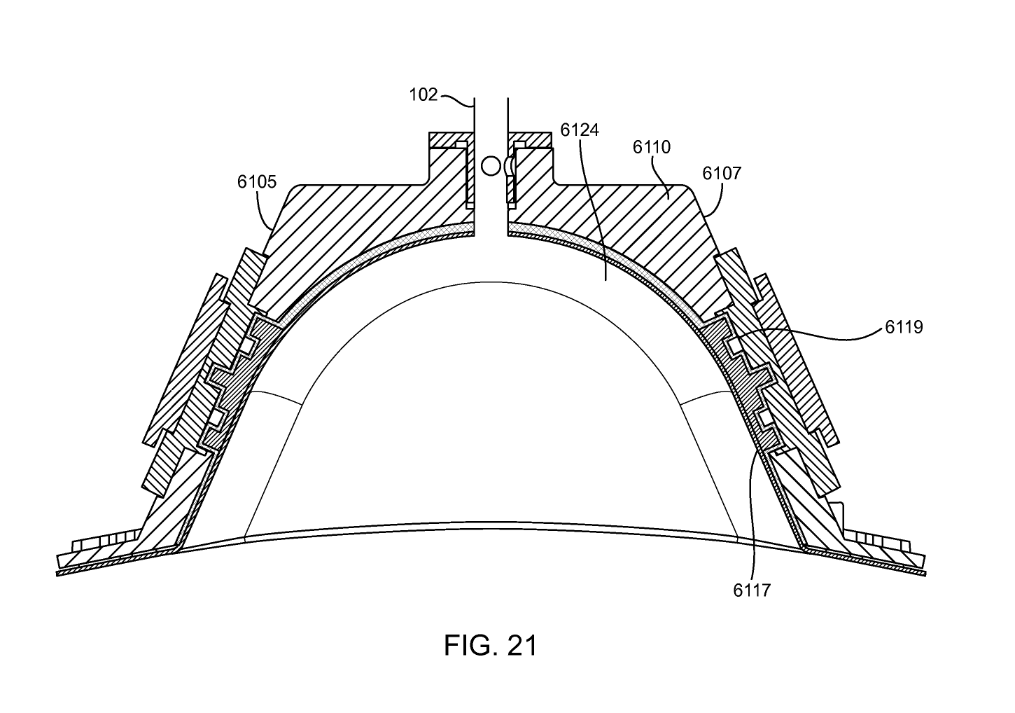

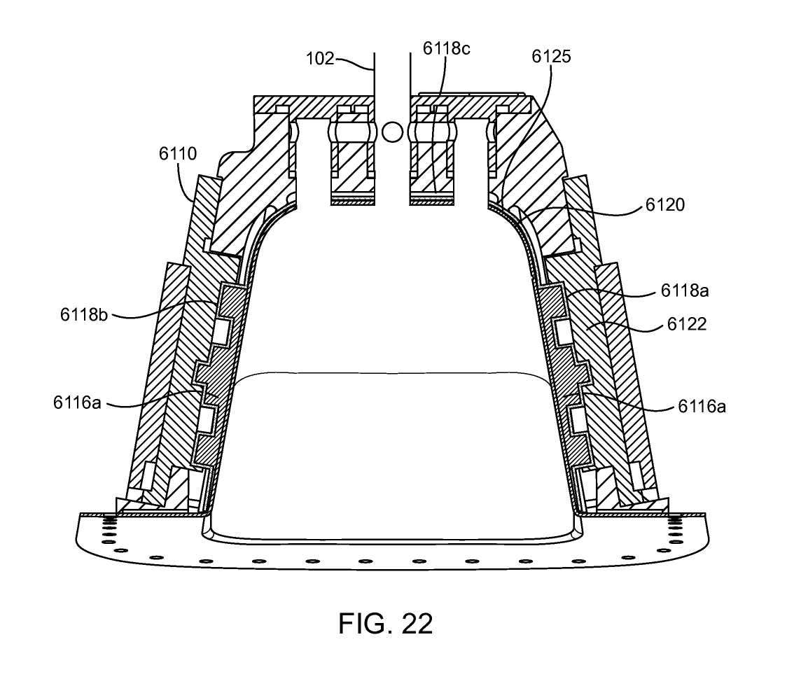

FIG. 21 is a cross sectional view of the applicator of FIG. 17 cut along the front plane 6112 of FIG. 17. FIG. 22 is a cross sectional view of the applicator of FIG. 17 cut along the Right Plane 6114 of FIG. 17

Within the housing 6110, a cooling plate 6120 forms a vacuum chamber 6124 configured to receive the tissue layers 200. The cooling plate 6120 may be constructed of any thermally conductive material or a dielectric material, for example, a plastic or ceramic. The vacuum input 6102 provides a vacuum from a vacuum source 102 to the vacuum chamber 6124 to draw tissue into the chamber 6124. Electrodes 6116a are attached within the housing 6110 and to the outer surface 6121 of the cooling plate 6120 and form a first pair of generally opposing electrodes having an arched shape similar to that of the front plane 6112 or side 6101 of the applicator 6100. Electrodes 6116b are attached within the housing 6100 to end 6105, 6107 of the cooling plate 6120 and form a second pair of generally opposing electrodes and shaped to follow the curved shape of the cooling plate structure 6120 from the first end 6105 to the second end 6107. The electrodes 6116a, 6116b are energized through wires 104.

The housing 6110 is spaced from electrodes 6116a 6116b, and the cooling structure 6120 forming a coolant chamber 6118 having a flow path 6119 from the inlet 6106 to the outlet 6108. The flow path 6119 of the coolant through the coolant chamber 6118 is defined by a plurality of flow chambers 6118a, 6118b, and 6118c in communication with the flow path 6119 from the inlet 6106 to the outlet 6108. First side flow chambers 6118a is defined by the housing structure 6110, ribs 6125 on the housing structure 6110, and the cooling plate 6120 which sealingly form a chamber 6118a extending from a first end 6105 of first side 6101 to the second end 6107 of the first side 6101. Similarly the second side flow chamber 6118b is defined by the housing structure 6110, ribs 6125, and cooling structure 6120 which sealingly form a chamber 6118b extending from the first end 6105 of the second side 6103 to the second end 6107 of the second side 6103. A third flow chamber 6118c extends over the arched surface 6123 of the cooling plate 6120 from the second end 6107 back to the first end 6105 of the applicator 6100. Ribs 6125 on the housing 6110 and the arched surface 6123 form the third flow chamber 6118c.

Coolant fluid 6122 from the coolant inlet 6106 flows first and equally through first and second side flow chambers 6118a and 6118b. After coolant 6122 flows from the inlets 6106 into chambers 6118a and 6118b from the first end 6105 to the second end 6107, flow of coolant 6122 is directed from the second end 6107 through the third chamber 6118c over the arched surface 6123 back to the first end 6105 where the coolant 6122 exits the outlet 6108. Inlets 6106 and outlet 6108 are isolated from each other on the first end 6105 by features 6123a on housing 6110. The flow of coolant 6122 through the cooling chamber 6118 while fat tissue is being heated serves to protect the outer layers of the skin while fat is being heated.

Electrodes 6116a, 6116b have a first dielectric interfacing surface 6117 and a back surface opposing the interfacing surface 6119. The back surface 6119 is exposed to the coolant 6122 flowing through the cooling chamber 6118. According to the invention, electrodes 6116a, 6116b or other electrodes described in iterations herein comprise flow channels 6115 that direct the flow of coolant 6122 within the flow chamber 6118 over the electrode surface 6119 to provide and increases cooling effect on the electrodes, e.g., electrodes 6116a, 6116b during use.

In accordance with the invention, the chambers 6118a, 6118b and 6118c may be isolated from each other and may have separate coolant inputs and outlets. Accordingly the coolant in each chamber may be cooled or heated to a different temperature.

The vacuum chamber 6124 further comprises an inner surface 6125 and fiber optic grooves 6126 in the inner surface 6125 directing fiber optic 6129 to potential hot zones 6128 within the chamber 6124. The fiber optic 6129 or other sensors may be used to detect when the specific hot spot has reached an undesired temperature. The device may be turned off or the power, frequency, or cooling settings may altered upon detection (either manually or automatically with a controller).

In addition, sensors 6130 may be provided to determine whether or not adequate tissue contact is made with the cooling plate 6120 and/or whether tissue acquisition into the vacuum chamber 6124 is at a desired level. A variety of possible sensors include but are not limited to the examples described herein. The sensors 6130 are shown located on the inside wall of the cooling plate 6120 in the vacuum chamber 6124. However, some sensors may alternatively be located on the outside of the cooling plate. Any of the sensors described herein including those shown in FIGS. 17-25 may be incorporated into any of the applicators illustrated or described herein.

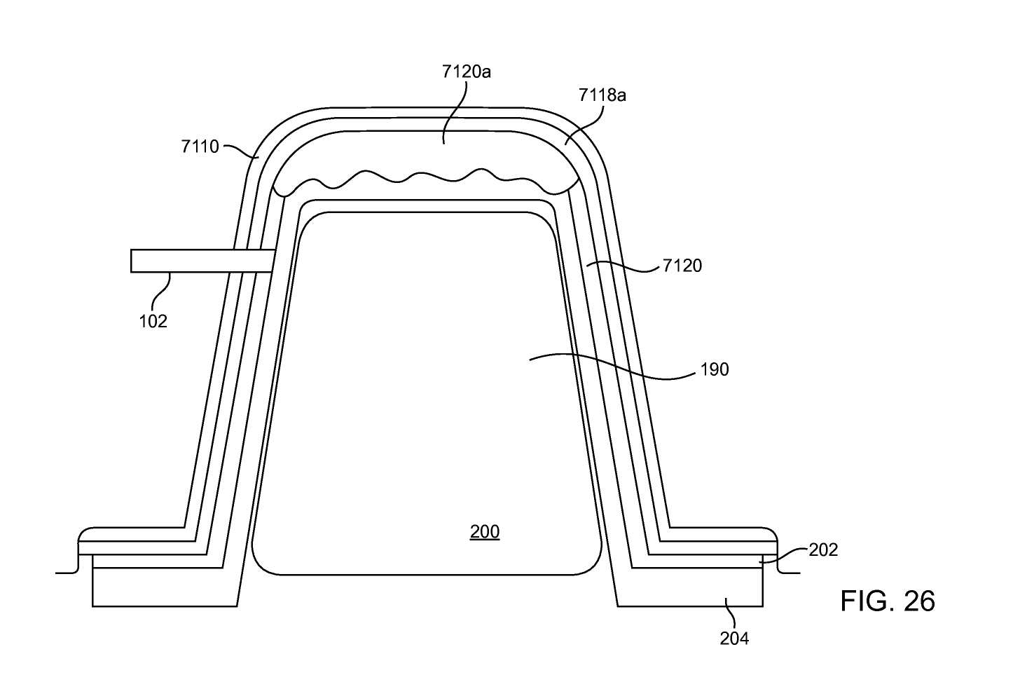

FIG. 26 illustrates a housing 7110 that may be used with any of the applicators, systems or methods described herein. The housing 7110 is shown with acquired tissue 200 including skin 202 and fat 204. The housing includes a cooling chamber 7118 between the housing 7110 and a cooling plate 7120. The cooling plate 7120 forms a vacuum chamber 7124 with a vacuum source 102. The top or ceiling 7120a of the cooling plate comprises a flexible material such as an elastomer. The flexible material is provided to increase skin 202 contact with the cooling plate 7120.

FIG. 26 also illustrates a therapeutic zone 7190 which is the zone at which energy is directed to heat to a therapeutic temperature for a period of time. Each of the applicators described herein are configured to direct energy to a desired target zone.

A method of transmitting energy to a patient for the purpose of reducing fat is provided. According to variations, radiofrequency energy is transmitted through opposing electrodes acting as capacitor plates to heat tissue. According to variations, energy is transmitted to fat through an applicator having an acquisition chamber for positioning tissue with respect to the electrodes to deliver desired heating energy, to heat fat tissue to a desired temperature and/or to reduce the volume of fat in the treated tissue. According to variations on the methods, tissue may be positioned with respect to the electrodes to reduce hot spots of temperature and/or to protect the skin. According to methods of the invention, applicators, sensors control consoles and systems may be used to reduce fat volume as described herein.

In use, an applicator as described herein may be positioned on the skin in a desired location. The procedure may be used on many body regions. The midsection, including the lateral sections (what are generally referred to as “love handles”) may be particularly opportunistic target regions. Once in place tissue is acquired into a chamber by applying a vacuum. Treatment is provided to the tissue using one or more of a variety of aspects of the applicators, sensors, feedback and control as described herein.

The following is an example of a protocol used in treatment of a subject in accordance with the invention.

### Example 1

In this example, an applicator constructed according to dimensions as shown in FIGS. 17-25 was applied to tissue of a subject. The height of the tissue acquisition chamber from the end to max height of the ceiling was about 2.58 inches. The height of the electrode on the applicator ends was about 1.06 inches. The highest point of the electrode on the ends was positioned about a distance of 0.76 inches from the max height of the ceiling of the acquisition chamber. Thus a gap was created between the electrode and the location of acquired skin that would be is near parallel with the electrode field. Similarly, the height of the tissue acquisition chamber from the mid point of the side to the max height of the ceiling was about 2.38 inches. The peak height of the electrode on the applicator side was about 1.38 inches. The highest point of the electrode on the ends was positioned about a distance of 0.63 inches from the max height of the ceiling of the acquisition chamber. Thus a gap was created between the electrode and the location of acquired skin that would be is near parallel with the electrode field.

Further according to the example an applicator was placed in the appropriate treatment position on the patient's skin and target location was confirmed.

1. Pre cool phase: Apply vacuum to acquire tissue. Visually confirm or using sensor, confirm skin is fully acquired inside the vacuum chamber of the applicator. Wait for a pre-cool period of time prior to energy delivery. In one iteration, skin was pre-cooled at 13 degrees C. for 7 minutes.

2. Ramp-up phase: Ramp up treatment to slowly increase temperature in the treatment zone. In one iteration, treatment was applied using a single electrode pair for 5 minutes at a net of 50 W. If a temperature feedback threshold is reached, then the subject is moved to the transition step. If temperature feedback threshold is not met then treatment is continued at 50 Watts until threshold is met.

3. Transition phase: During the transition phase, treatment is alternated between electrode pairs. In iteration, treatment was applied at alternative 5 second intervals at 20 Watts of power for a period of 2 minutes. Coolant temperature was raised to 31 degrees C. Transition phase may be increased or decreased in duration depending on feedback from subject or sensors

4. Steady State Phase: During the steady state phase, power is reduced to 10 Watts and is applied for an additional 15 to 30 minutes. Depending on feedback, the power may be reduced for a period of time and then resumed. A transition phase may be resumed after the power has been turned off and then a steady state is reached.

In situations where it is desirable to treat a large volume of tissue or where it is desirable to treat multiple locations at one time, multiple applicators could be used in parallel.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. An applicator for use in the reduction of fat, the applicator comprising:
a housing containing:
a tissue acquisition chamber having an internal surface adapted to contact tissue;
a cooling plate, wherein the cooling plate forms at least a portion of the internal surface of the acquisition chamber;
a coolant chamber adjacent at least a portion of the cooling plate;
at least one electrode pair comprising electrodes positioned on opposing sides with respect to the tissue acquisition chamber; and
a tissue acquisition structure configured to bring tissue into the tissue acquisition chamber.

2. The applicator of claim 1 wherein the electrodes are separated from the acquisition chamber by the cooling plate.

3. The applicator of claim 1 wherein the electrodes form at least a portion of the internal surface of the acquisition chamber.

4. The applicator of claim 1 wherein cooling plate comprises a cutout and wherein an electrode of the electrode pairs is positioned within the cutout.

5. The applicator of claim 1, wherein the electrodes are separated from the cooling plate by the coolant chamber.

6. The applicator of claim 1, wherein the coolant chamber includes a cooling fluid moving through the coolant chamber.

7. The applicator of claim 1, wherein the electrodes are coupled to a wall of the cooling plate opposite the wall which forms the internal surface.

8. The applicator of claim 1 wherein the coolant chamber is positioned adjacent the cooling plate and the electrodes.

9. The applicator of claim 1 wherein the tissue acquisition chamber comprises a vacuum tube configured to be coupled to a vacuum source and connected to the tissue acquisition chamber.

10. The applicator of claim 1 wherein the electrodes include a coolant channel formed therein and are configured to receive a coolant flowing through the coolant channel.

11. The applicator of claim 1 wherein the electrodes forming a first of the at least one electrode pairs are substantially parallel with respect to each other.

12. The applicator according to claim 1 wherein the electrodes of the first electrode pair form an angle with respect to each other of between 45 and 0 degrees.

13. The applicator of claim 1 wherein at least one of the electrodes is movable within the housing from a first configuration to a second configuration.

14. The applicator of claim 13 wherein the second configuration comprises an angular orientation with respect to the first configuration.

15. The applicator of claim 1 wherein the electrodes comprise a plurality of electrode pairs.

16. The applicator of claim 1 wherein the electrodes further comprise a second pair of electrodes, wherein the electrodes of the first electrode pair have a first dimension and the electrodes of the second electrode pair have a second dimension.

17. The applicator of claim 16 wherein the dimension comprises one or more of length, width size, shape, surface area, and volume.

18. The applicator of claim 15 wherein the acquisition chamber internal surface comprises a first side and an opposing second side, and a third side and an opposing fourth side, wherein the plurality of electrode pairs comprises a first electrode pair including a first electrode positioned adjacent the first side of the acquisition chamber and a second electrode positioned adjacent the second side of the acquisition chamber, and wherein the second electrode pair comprises a third electrode positioned adjacent the third side of the acquisition chamber and a fourth electrode positioned adjacent the fourth side of the acquisition chamber.

19. The applicator of claim 1 wherein tissue acquisition chamber forms a tissue treatment zone when tissue is acquired into the chamber.

20. The applicator of claim 19 wherein the electrodes are positionable to form a therapeutic treatment zone of tissue acquired within the acquisition chamber.

21. The applicator of claim 1 comprising a plurality of coolant chambers.

22. The applicator of claim 21 wherein each of the plurality of coolant chambers separate from the other of the coolant chambers.

23. The applicator of claim 19 further comprising a sensor configured to sense a temperature of tissue outside of the treatment zone.

24. The applicator of claim 1 comprising one or more sensors configured to sense contact of skin with the acquisition chamber internal surface at one or more locations on the acquisition chamber internal surface.

25. The applicator of claim 1 wherein the at least a portion of the acquisition chamber inner surface comprises a flexible material configured to conform tissue when it is acquired into the chamber.

26. The applicator of claim 1 wherein the internal surface of the acquisition chamber comprises a first side and second opposing side opposing the first side wherein the first and second sides form an angle with respect to each other of greater than 0 degrees.

27. The applicator of claim 1 wherein the internal surface of the acquisition chamber comprises a first side and second opposing side opposing the first side wherein the first and second sides form an angle with respect to each other of greater than 45 degrees.

28. The applicator of claim 1 wherein the acquisition chamber internal surface comprises a first side and an opposing second side, and a third side and an opposing fourth side, wherein the plurality of electrode pairs comprise a first electrode positioned adjacent the first side of the acquisition chamber and a second electrode positioned adjacent the second side of the acquisition chamber, wherein the first side forms a first angle with respect to the second side and the third side forms a second angle with respect to the fourth side, wherein the second angle is greater than the first angle.

29. The applicator of claim 28 wherein the first angle is less than or equal to 45 degrees.

30. A system for use in the reduction of fat comprising:
a housing containing:
a tissue acquisition chamber having an internal surface adapted to contact tissue;
a cooling plate, wherein the cooling plate forms at least a portion of the internal surface of the acquisition chamber;
a coolant chamber adjacent at least a portion of the cooling plate;
at least one electrode pair comprising electrodes positioned on opposing sides with respect to the tissue acquisition chamber; and
a tissue acquisition structure configured to bring tissue into the tissue acquisition chamber;
a control console comprising:
a coolant source configured to circulate a coolant through the coolant chamber an RF generator configured to be coupled to the plurality of electrodes; and
a controller configured to control the delivery of an RF signal to the plurality of electrodes.

31. A method of body contouring comprising:
bringing tissue comprising skin and fat into an acquisition chamber of an applicator;
contouring a volumetric size and shape of a therapeutic zone of tissue by selectively heating and cooling using at least one electrode pair positioned in the applicator on opposing sides of the acquisition chamber.
